(19) United States
(12) Patent Application Publication
(10) Pub. No.: US 2008/0124756 A1
(43) Pub. Date: May 29, 2008

(54) Light utilization alteration of photosynthetic microorganisms

(75) Inventor: Harrison Dillon, Palo Alto, CA (US)

Correspondence Address: Solazyme, Inc., 3475-T Edison Way, Menlo Park, CA, 94025, US

(73) Assignee: Solazyme, Inc., Mountain View, CA (US)

(21) Appl. No.: 10/934,228

(22) Filed: Sep. 3, 2004

(51) Int. Cl. C12P 21/06; C12N 1/12; C12N 15/74

(52) U.S. Cl. 435/69.1; 435/471; 435/257.2

(57) ABSTRACT

Methods provided herein are directed to increasing the efficiency of light utilization of photosynthetic microorganisms. Also provided are screening assays, genetic constructs, and photosynthetic microorganisms for increasing light utilization efficiency and production of molecules such as ATP, oxygen, hydrogen, and recombinant proteins. Methods provided herein can be performed with any photosynthetic microorganism, including prokaryotic and eukaryotic microorganisms.

### BACKGROUND OF THE INVENTION

Photosynthetic microorganisms turn light energy into chemical energy through a series of biochemical reactions. Light energy, in the form of photons, is absorbed by light harvesting antennas associated with two large, transmembrane complexes known as Photosystem I (PSI) Photosystem II (PSII). Photons are absorbed by pigment molecules in the antenna and core PSI and PSII complexes. In PSII, the energy absorbed by a pigment molecule such as chlorophyll a or chlorophyll b is transferred via other pigment molecules to the reaction center, where a cluster of four manganese atoms participates in the splitting of two water molecules into dioxygen and reducing equivalents. Electrons removed from the water molecules are routed through the photosynthetic electron transport chain, which consists of PSII, the cytochrome b6f complex, and PSI. This transfer of electrons is fueled by energy absorbed by photons. In addition to pigments embedded in the core PSI and PSII complexes, pigments are also embedded in peripheral antenna complexes. These peripheral antenna complexes harvest photons and direct the harvested energy toward the PSI and PSII core complexes.

Under-high light conditions, the peripheral antenna complexes harvest more photons than can be effectively routed through the electron transport chain. The extra energy from these photons is dissipated as heat. The heat dissipation mechanism allows the cells to avoid deconstructing the light harvesting antennas when bright light is available.

### BRIEF SUMMARY OF THE INVENTION

Provided herein are methods of generating a desired phenotype in a photosynthetic microorganism comprising transforming the microorganism with at least one light utilization alteration construct, wherein a light utilization alteration segment within the light utilization alteration construct is in operable linkage with a light activated promoter; and screening or selecting for the desired phenotype in the presence of light. In some methods at least part of the nucleotide sequence of the light activated promoter is within 3000 base pairs of the start codon of a gene selected from Table 2. In some methods a plurality of microorganisms is transformed with a plurality of light utilization alteration constructs and resulting transformants are individually screened for the desired phenotype. In some methods the light activated promoters are generated by amplifying staggered lengths of one or more light activated promoters. In some methods the light activated promoter is generated by error-prone amplification. In some methods the light activated promoter contains nucleotide sequence from the promoter of more than one gene. In some methods the light utilization alteration segment comprises at least 10 nucleotides of a gene that encodes a protein that binds at least one light absorbing pigment, or a protein that catalyzes biosynthetic production of light absorbing pigment molecules a protein that modulates photosynthetic activity through signal transduction, or a protein that dissipates absorbed light energy as heat. In some methods the light utilization alteration segment comprises at least 10 nucleotides of a gene that encodes a protein listed in Table 1. In some methods the light utilization alteration segment comprises at least 10 nucleotides of a gene that encodes a protein that has at least 50% amino acid sequence identity with a protein listed in Table 1.

In some methods the desired phenotype is a higher level of oxygen evolution than that of a starting strain. In other methods the desired phenotype is a higher level of ATP production than that of a starting strain. Some methods further comprise identifying a transformed microorganism that generates an increased amount of ATP over a starting strain. Still further methods comprise transforming an identified microorganism with at least one gene encoding an enzyme that participates in the synthesis of a molecule from the list consisting of a hydrocolloid, isoprenoid, polyketoid, fatty acid, lipid, carotenoid, polysachharide, or antibiotic molecule and/or with at least one gene encoding a recombinant human protein selected from the list consisting of insulin, interferon alpha, erythropoietin, human growth hormone, granulocyte-colony stimulating factor, tissue plasminogen activator, a human immumoglobulin and Factor VIII. In other methods the desired phenotype is a higher level of hydrogen production than that of a starting strain. In other methods the desired phenotype is a higher level of production of a recombinant protein than that of a starting strain.

In some methods the screening or selecting takes place in at least 10 μmol photon m⁻² s⁻¹. In other methods the screening or selecting takes place in at least 100 μmol photon m⁻² s⁻¹. In other methods the desired the screening or selecting takes place in at least 1000 μmol photon m⁻² s⁻¹. In other methods the desired the screening or selecting takes place in at least 1500 μmol photon m⁻² s⁻¹.

In some methods a plurality of microorganisms are screened or selected after being arrayed into microtiter plates made of non-transparent material. In some methods the microorganism is eukaryotic. In some methods the microorganism is of a genus selected from the group consisting of Chlamydomonas, Chlorella, Volvox, Phaeodactylum and Thalassiosira. In some methods the microorganism is Chlamydomonas reinhardtii. In some methods the microorganism is Chlorella vulgaris or Chlorella ellipsoidea. In some methods the microorganism is Phaeodactylum tricornutum. In some methods the microorganism is Thalassiosira weissflogii.

In some methods the microorganism is prokaryotic. In some methods the microorganism is of a genus selected from the group consisting of Thermosynechococcus, Synechococcus, Anabaena, Synechocystis, and Fremyella. In some methods the microorganism is Thermosynechococcus elongates. In some methods the microorganism is Synechococcus PCC 7942. In some methods the microorganism is Anabaena PCC 7120. In some methods the microorganism is Synechocystis sp. PCC 6803 or Synechocystis sp. BO8402. In some methods the microorganism is Fremyella diplosiphon. In some methods the microorganism is listed in Table 4.

In some methods measurement of ATP is performed by measuring light output from a luciferase protein encoded by a luciferase gene present in a genome of the microorganism. In some methods measurement of ATP is performed by measuring light output from a luciferase protein added to cells before, during, or after lysis. In some methods the microorganism is eukaryotic and the luciferase gene is in the chloroplast genome.

Methods are provided for increasing the utilization efficiency of absorbed light energy in a photosynthetic microorganism incapable of flagella-based motility comprising transforming the microorganism with an RNAi construct in operable linkage with a light activated promoter, wherein the RNAi construct targets a transcript encoding an antenna protein in the microorganism; culturing the transformed microorganism in a culture container made of non-transparent material; exposing the transformed microorganism to light only from above the plane of the surface of the culture media; and screening the transformed microorganism for the ability to generate more oxygen, hydrogen, recombinant protein or ATP than a starting strain.

Photosynthetic microorganism are provided containing an antisense or RNAi construct that targets a transcript of a gene that encodes a protein involved in light harvesting, wherein the antisense or RNAi construct is in operable linkage with a promoter that is activated by light.

Genetic constructs are provided comprising a light activated promoter; an antisense or RNAi segment that contains at least 10 nucleotides of a gene encoding a protein involved in light harvesting; and a screenable or selectable marker gene in operable linkage with a promoter. In some genetic constructs an antisense or RNAi segment encodes a section of a gene that encodes a protein that binds a light absorbing pigment.

Also provided are populations of photosynthetic microorganisms in liquid culture media, wherein: the population is exposed to light from above the plane of the surface of the culture media; at least one cell in the population contains an antisense or RNAi segment comprising at least 10 nucleotides of a gene encoding a protein involved in light harvesting in operable linkage with a promoter that is activated by light; and cells on the top of the population express the antisense or RNAi segment at a higher level than cells on the bottom of the population In some populations the cells of the population are incapable of flagella-based motility.

Also provided are methods of producing a cell with a desired phenotype comprising generating a plurality of promoter segments by amplifying a plurality of distinct regions of a promoter of at least one gene; placing at least one genetic construct to be expressed in operable linkage with a member of the plurality of promoter segments to create a library of differentially induced genetic constructs; transforming a population of cells with the library; and screening or selecting for the desired phenotype.

Also provided are methods of increasing utilization efficiency of absorbed light energy in a C. reinhardtii cell comprising expressing an RNAi construct encoding an antenna gene in a C. reinhardtii cell through operable linkage with a light activated promoter; culturing the cell in a culture container made from non-transparent material; screening for hydrogen production under conditions wherein light is provided to the culture container from above.

Provided herein are methods of generating a library of promoters comprising amplifying at least two distinct segments of at least one promoter, wherein each distinct segment is amplified by a first primer that contains a region at its 5′ end that is not complementary to any promoter sequence being amplified; and a second opposing primer that contains the complement of the region at its 5′ end; denaturing the at least two distinct segments; annealing the at least two segments to generate a concatamerized assembly of distinct segments; and extending the assembly with a polymerase.

In some methods a light utilization segment encodes an RNAi molecule. In some methods the RNAi molecule targets transcripts from more than one gene. In some methods a light utilization segment encodes an antisense molecule. In some methods the antisense molecule targets transcripts from more than one gene. In some methods a light utilization segment encodes an antibody gene, wherein the antibody encoded by the gene specifically binds a protein involved in light harvesting.

### BRIEF DESCRIPTION OF THE DRAWINGS

FIG. 1 shows a schematic diagram of exemplary light utilization alteration constructs with examples of various components. FIG. 1 also shows an example of synthesis of a stem-loop construct.

FIG. 2 shows an example of a method of generating a combinatorial library of light utilization alteration constructs.

FIG. 3 shows an example of an RNAi light utilization alteration segment targeting a C. reinhardtii gene.

FIG. 4 shows an example of an antisense light utilization alteration segment targeting a Synechococcus gene.

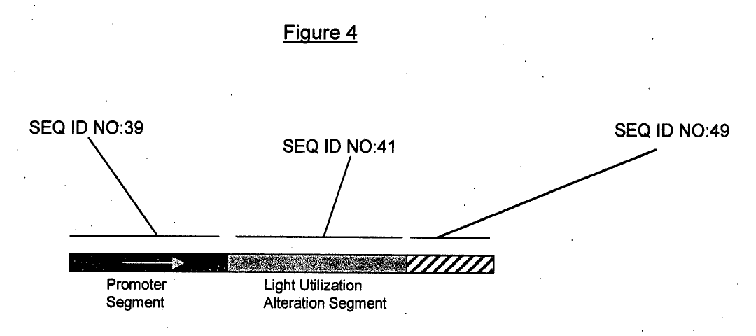

FIG. 5 shows a comparison of photosystem II antenna amounts in cells as a function of depth of culture in wild type strains versus light harvesting optimized strains.

FIG. 6 shows a comparison of photosystem I antenna amounts in cells as a function of depth of culture in wild type strains versus light harvesting optimized strains.

FIG. 7 shows a codon-shifted protein encoding light utilization alteration construct and a constitutive antisense expression construct for coexpression in a photosynthetic microorganism.

FIG. 8 shows an example of an amplification strategy for generating staggered promoter fragments of the C. reinhardtii Mg chelatase ChlI subunit gene promoter.

FIG. 9 shows the promoters of the C. reinhardtii Mg chelatase ChlI subunit gene promoter and phosphoglycerate kinase gene promoters.

FIG. 10 shows a photosynthesis assay measuring oxygen evolution using transition metal containing chemochromic films.

FIG. 11 shows a comparison of chlorophyll/cell amounts as a function of depth of, culture in wild type strains versus light harvesting optimized strains.

FIG. 12 shows an example of a light utilization alteration construct designed to paralyze a Synechococcus starting strain and integrate the construct into the genome.

FIG. 13 shows an example of a design of a combinatorial light utilization alteration construct library.

### DETAILED DESCRIPTION OF THE INVENTION

Definitions: The following definitions are intended to convey the intended meaning of terms used throughout the specification and claims, however they are not limiting in the sense that minor or trivial differences fall within their scope.

“Light utilization alteration construct” means a genetic construct comprising at least (1) a light utilization alteration segment in operable linkage with a promoter and (2) a screenable or selectable marker gene in operable linkage with a promoter. “Light utilization alteration segmnent” means a nucleic acid containing at least nucleotides that are identical to a segment of a gene encoding a protein involved in light harvesting. “Protein involved in light harvesting” means a protein that (1) binds at-least one light absorbing pigment molecule; or (2) catalyzes biosynthetic production of light absorbing pigment molecules; or (3) modulates photosynthetic activity through signal transduction; or (4) dissipates absorbed light energy as heat; or (5) specifically binds a protein from groups 1-4. Examples of each group are (1) phycobilisome core protein from Synechocystis sp. PCC 6803; (2) magnesium chelatase from Chlorella vulgaris; (3) tlaI from Chlamydomonas reinhardtii; (4) Lhcbm1 from Chlamydomonas reinhardtii; and (5) an antibody that binds the tla1 protein from Chlanydomonas reinhardtii. The groups are not necessarily mutually exclusive.

“Operable Linkage” means linkage in which a regulatory DNA sequence such as a promoter and a DNA sequence sought to be expressed, such as a cDNA, antisense or RNAi construct, are cornected in such a way as to permit expression. A transcriptional termination sequence can also be placed in operable linkage with a DNA sequence sought to be expressed to permit transcriptional termination.

“Starting Strain” means a strain that has not been transformed with a light utilization alteration construct.

A “codon shifted protein-encoding segment” is a cDNA that encodes a protein involved in photosynthesis using different codons than the endogenous version of the gene that encodes the protein involved in photosynthesis in a photosynthetic microorganism.

A “heterologous promoter” is a promoter that is placed in operable linkage with a nucleic acid sequence sought to be expressed that is different from the promoter that is in operable linkage with the nucleic acid in a wild-type organism.

The term “modulation” when used in the specification in a context such as “targets for modulation using light utilization alteration constructs” means: increasing or decreasing the amount of a protein involved in light harvesting using a light utilization alteration construct in a photosynthetic microorganism under a given light intensity, compared to the photosynthetic microorganism not transformed with the light utilization alteration construct under the same light intensity.

“Flagella-based motility” means the ability of a cell to move within an aqueous environment through the use of flagella Cells can be deficient in flagella-based motility due to a natural lack of flagella or through mutagenesis.

“Light absorbing pigment” means a molecule that is bound by a protein in physical association with a photosystem complex Examples include chlorophyll a, chlorophyll b, lutein, β-carotene, zeaxanthin, and lycopene.

“RNAi stem loop” means a nucleic acid molecule in which a first region of the molecule contains a nucleotide sequence that is complementary with a second region of the same molecule, wherein the first and second regions are separated by a third region that is not complementary to the first or second regions.

The term “endogenous” refers to a gene in that is present in a wild type organism or a protein that is produced by translation of a transcript that is transcribed from a gene that is present in a wild type organism.

“Light activated promoter” means any nucleic acid sequence that activates transcription in a cell in response to light.

A protein that “modulates photosynthetic activity” causes a change in the level of photooxidative water splitting activity when its cellular concentration is increased or decreased.

“Culture media” means any substrate, liquid or solid, that a photosynthetic microorganism can grow in. Culture media is not limited to a substrate generated by a practitioner (such as Sager's minimal media or BG11 media, for example), and includes seawater, freshwater, brackish water, and any of the foregoing that has been altered by the addition or removal of components from the substrate.

A protein such as an antibody “specifically binds” another molecule when the protein functions in a binding reaction which is determinative of the presence of the molecule in the presence of a heterogeneous population of molecules. Thus, under designated immunoassay conditions, the specified protein binds preferentially to a particular molecule and does not bind in a significant amount to other molecules present in the sample. Solid-phase ELISA immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity.

The term “amino acid sequence identity” means that two protein sequences, when optimally aligned, such as by the programs GAP or BESTFIT using default gap weights, share a specified percentage of the total number of amino acids in the sequences. For sequence comparison to determine the level of amino acid sequence identity, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFFIT, FASTA, and TFASTA in the Wisconsin Genetics Software Package, Genetics Computer Group, 575 Science Dr., Madison, Wis.), or by visual inspection (see generally Ausubel et al, supra). One example of algorithm that is suitable for determining percent sequence identity and sequence similarity is the BLAST algorithm, which is described in Altschul et al, J. Mol. Biol. 215:403-410 (1990). Software for performing BLAST analyses is publicly available through the National Center for Biotechnology Information (http://www.ncbi.nlm.nih.gov/). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff& Henikoff, Proc. Natl. Acad. Sci. USA 89,10915 (1989)).

U.S. patent application Ser. Nos. 10/287,750, 10/763,712, 10/411,910 and 60/500,032 are hereby incorporated by reference in their entirety for all purposes.

This application claims priority to U.S. patent application Ser. No. 60/500,032.

### I General

Methods are provided for increasing the efficiency of conversion of light energy into chemical energy by a population of photosynthetic microorganisms. At a given latitude in outdoor conditions, or under constant artificial light indoors, a certain number of photons hit a square unit of area. When photons hit a bioreactor containing photosynthetic microorganisms, some of the photons are converted into chemical energy. At the theoretical maximum level of conversion, every photon is utilized by photosynthetic microorganisms for conversion into chemical energy. In practice less than the theoretical maximum level of conversion occurs. When relatively bright light shines on a bioreactor containing photosynthetic microorganisms, the antenna complexes of photosynthetic microorganisms on the top layers of the culture harvest more photons than they can utilize. The excess photon energy is dissipated as heat. Cells underneath the top layers are shaded by the cells above since many of the photons that hit the bioreactor are absorbed and dissipated by the top layers of cells. The result is that a bioreactor containing a population of wild type photosynthetic microorganisms does not efficiently turn light energy into chemical energy because many of the photons absorbed by the cells in the top layers are not utilized for creation of chemical energy.

Methods are provided for increasing the efficiency of conversion of photons into chemical energy by a population of photosynthetic microorganisms. Some methods work by transformation of one or more starting strains of photosynthetic microorganisms with light utilization alteration constructs that downregulate expression of target genes that encode proteins involved in light harvsting in response to light. In some methods the starting strain is a wild type strain, while in other methods the starting strain has been genetically transformed to have an altered phenotype such as reduced motility. In some methods the downregulation is achieved through expression of an RNAi or antisense molecule by a light-induced promoter. Examples of genes encoding proteins involved in light harvesting are PSI and PSH antenna genes such as Lhca2 and Lhcbm4, respectively, chlorophyll biosynthesis genes such as hydroxymethylbilane synthase, and signaling genes such as tiaI. Photosynthetic microorganisms transformed with light utilization alteration constructs are placed in containers that allow light to strike the cells only from above the plane of the surface of the culture media. In one embodiment this is accomplished by culturing transformed cells in multiwell plates made of non-transparent plastic. The cells are preferably cultured in minimal media that requires the cells to grow photoautotrophically. Light is directed to the cells, preferably from directly above. A cellular function that requires energy is then assayed. Novel strains that perform the energy requiring function more effectively than the starting strain are identified through a screening or selection protocol.

In other methods genes involved in photosynthesis are inactivated in the genome of a starting strain and are re-introduced under the control of a heterologous promoter. The heterologous promoter is preferably activated by dark or low light conditions but not high light conditions. For example, the tial gene is downregulated through constitutive expression of an RNAi molecule targeting the tial transcript from a first expression vector. A synthetic gene encoding the tlal protein, but using different codons than the endogenous gene, is expressed from a heterologous promoter that is activated by darkness or by weak light but not bright light from a second expression vector. Encoding the synthetic gene using codons that differ from the wild type gene but do not alter the sequence of the protein encoded by the gene allows the transcript produced by the synthetic gene to avoid targeting by the RNAi molecule that directs degradation of the wild type transcript. Preferably the different codons used in the synthetic gene are preferred codons of the host organism. The net effect on cells through coexpression of the first and second constructs is a decrease in the amount of tlal protein in cells exposed to bright light and an increase in the amount of tlal protein in cells exposed to weak light.

The methods provided herein generate novel strains of photosynthetic microorganisms that have enhanced light utilization efficiency phenotypes. These novel strains dissipate less heat than wild type strains under bright light conditions. The cells on the top layers absorb less light than starting strains, which allows more light to travel into middle layers of cells, reducing shading of the middle and lower layers. The increased number of photons that penetrate the middle and bottom layers of cells are converted into chemical energy. The increased conversion of light energy into chemical energy by the novel strains is detected by screening for generation of a molecule that requires energy to produce, such as adenosine triphosphate (ATP), oxygen molecules formed from the photooxidation of water molecules by photosystem II, hydrogen molecules, carotenoids, and recombinant proteins such as human insulin.

The novel strains provided are more effective at conversion of light energy into chemical energy under a given amount of light than starting strains. The methods and compositions described herein can be used to alter the light harvesting properties of both unicellular and multicellular eukaryotic photosynthetic microorganisms, such as C. reinhardtii and Volvox cartei, respectively, as well as prokaryotic photosynthetic microorganisms, such as Anabaena PCC7120 and Synechococcus sp. WH8102.

II Light Utilization Alteration Constructs for Transforming Photosynthetic Microorganisms

### A. Overall Design of Constructs

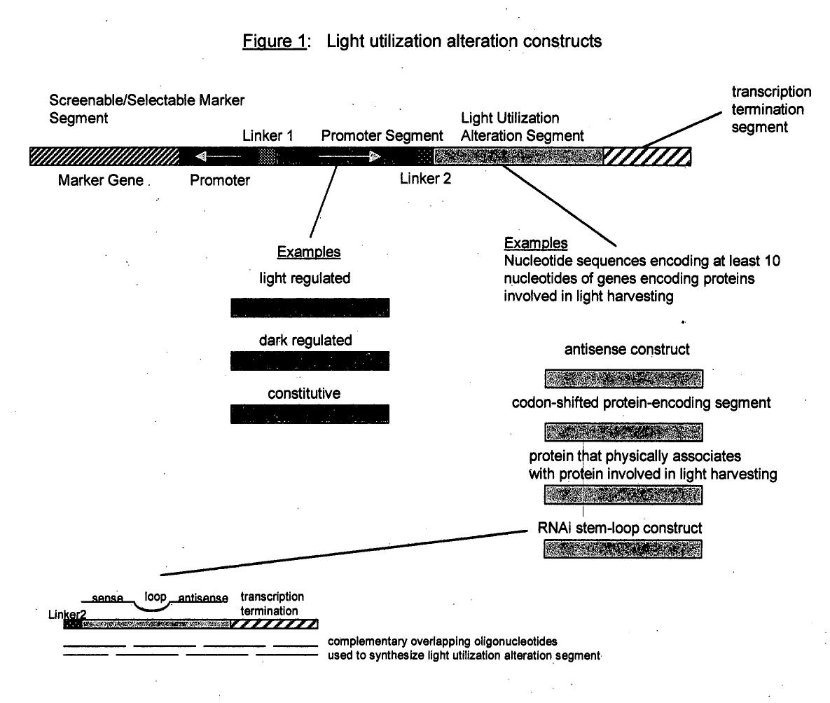

Light utilization alteration constructs are constructed by placing components of the constructs in operable linkage with each other. Examples of components of a light utilization alteration construct are a promoter segment, a light utilization alteration segment (such as an RNAi segment or a codon shifted protein-encoding segment), a transcription termination segment, linker segments, and a screenable or selectable marker containing a promoter in operable linkage with a marker gene. Other components can also be included in the constructs. Examples of light utilization alteration construct design are shown in FIG. 1.

### B. Light Utilization Alteration Segment

A light utilization alteration segment comprises a nucleic acid molecule that contains at least 10 nucleotides of a gene encoding a protein involved in light harvesting. In other embodiments the segment comprises at least 15, 18, 20, 25, 30, 40, 50, 75, 100, or more ucleotides of a gene encoding a protein involved in light harvesting. In some instances the segment encodes an RNAi stem-loop molecule. In other instances the segment encodes a sequence that is transcribed and translated, forming a protein, such as a codon shifted protein-encoding molecule. In other instances the segment encodes an antisense segment. Expression of the light utilization alteration segment in a population of photosynthetic microorganisms can alter the amount of incident photon energy that is converted into chemical energy by the cells under a certain light intensity.

If the light utilization alteration segment is an RNAi or antisense segment, light utilization is altered through the decreased amount of a protein involved in light harvesting produced by transcripts that are targeted for degradation by the RNAi or antisense molecule encoded by the light utilization alteration segment. In this instance the sequence identity between the RNAi or antisense segment and a transcript encoding a protein involved in light harvesting causes an expressed RNAi or antisense molecule to target the transcript. RNAi and antisense molecules target transcripts for degradation when there is usually at least 90% sequence identity between the molecule and a transcript. The RNAi or antisense segment is preferably in operable linkage with a promoter that is activated by light.

If the light utilization alteration segment encodes a protein, light utilization can be altered through functional light harvesting changes caused by the interaction of the protein with other molecules involved in photosynthesis. For example, expression of a monoclonal antibody that specifically binds to the tlal protein can alter light utilization in a cell. In addition, a codon shifted protein-encoding segment in operable linkage with a dark-activated promoter coexpressed with an RNAi molecule targeting the naturally occurring transcript of the gene encoding the protein can alter light utilization in a cell.

i. RNAi and Antisense Segments

RNAi segments are nucleic acid sequences that encode an RNAi molecule that generates a stem-loop structure, as shown in FIG. 1. RNAi molecules specifically recognize RNA transcripts that contain identical or substantially identical sequences and target them for degradation Targeting transcripts with RNAi molecules is a highly effective method of reducing the amount of a particular protein in a cell without altering the expression level of the gene that encodes the protein. RNAi molecule design is known and is described in the literature (see Cell, 2004 Apr. 2;117(1): 1-3; Proc Natl Acad Sci U S A. 2004 Apr. 13;101(15):5494-9; and Proc Natl Acad Sci USA. 2004 May 18;101(20):7787-92). The stem is preferably 5-500 base pairs in length, more preferably 15-50 base pairs in length, and more preferably 20-30 base pairs in length, and more preferably 21-25 base pairs in length. RNAi molecules encode a sense and antisense region of a gene to form the double stranded stem, most preferably a coding region, linked by a single stranded loop structure.

RNAi and antisense molecules have been demonstrated to eliminate or significantly reduce transcript numbers of genes in photosynthetic microorganisms (see for example J Cell Sci. 2001 November; 114(Pt 21):3857-63; Proc Natl Acad Sci U S A 2004 May 18;101(20):7787-92; Dev Cell, 2004 Mar. 6(3):445-511) RNAi segments described herein are designed to target transcripts of genes encoding proteins involved in light harvesting in photosynthetic microorganisms. These segments are designed by selecting a first “sense” region of a gene encoding a protein involved in light harvesting, such as a 25 base pair region that corresponds to a coding region of a gene. A second “loop” region that does not correspond to the first sequence or its complement is then added to the end of the sense region, as shown in FIG. 1. A third “antisense” region that is complementary to the first sense region is then to the end of the loop region. The resulting stem-loop sequence can be chemically synthesized as a single oligonucleotide or as a series of overlapping oligonucleotides in operable linkage with a transcription termination segment, as shown in FIG. 1.

In addition to RNAI stem loop structures, transcripts can also be targeted for degradation using antisense expression. An antisense molecule is a single stranded RNA molecule that is complementary to an RNA transcript. Expression of antisense constructs is an effective means to downregulate the production of a specific protein, and can be used in eukaryotic systems (Chen and Melis, Localization and finction of SulP, a nuclear-encoded chloroplast sulfate permease in Chlamydomonas reinhardtii, Planta, published online Jul. 24, 2004; J Cell Sci. 2002 Apr. 1;115(Pt 7):1511-22; Plant Cell. 1999 Aug. 11(8):1473-84) and prokaryotic systems (J Mol Biol. 1999 Dec. 17;294(5):1115-25; Oligonucleotides. 2003; 13(6):427-33; J Mol Biol. 2003 Nov. 7;333(5):917-29); EMBO J. 1994 Mar. 1;13(5):103947; Annu. Rev. Biochem. 1991, 60, 631-652; Annu. Rev. Microbiol. 1994, 48, 713-742; Antisense RNA structure and function, In RNA Structure and Function (1997), Cold Spring Harbor Laboratory Press, Cold Spring Harbor, N.Y.).

Some distinct genes encoding proteins involved in light harvesting have a high level of nucleotide identity with each other. Transcripts encoded by genes that are completely identical over a 20-25 base pair region or are almost completely identical (such as 20 of 22 base pairs in a region) can be targeted by the same RNAi or antisense molecule. Genes from the same gene family are candidates for targeting by the same RNAi or antisense molecule, such as the light harvesting peptides that comprise the LHCH antenna complex For example, the C. reinhardtii Lhcbm1 and Lhcbm2 cDNA sequences (GenBank Accession numbers 15430565 and 15430563, respectively) contain sections in excess of 25 nucleotides that have 100% sequence identity.

Because of the high level of sequence identity between genes of the same family that encode proteins involved in light harvesting, expression on a single antisense or RNAi construct can degrade transcripts from a plurality of antenna genes. For example, the nucleotide sequence ggccccaaccgcgccaagtggctgggccctac (SEQ ID NO:61) is found in the C. reinhardtii Lhcbm3, Lhcbm4, Lhcbm6, and Lhcbm9 genes. The nucleotide sequence tacctgactggcgagttccccgg (SEQ ID NO:31) is found in the C. reinhardtii Lhcbm1, Lhcbm2, Lhcbm3, and Lhcbm4, Lhcbm5, Lhcbm6, Lhcbm8, and Lhcbm9 genes. Many other segments of different genes that encode proteins involved in light harvesting are identical at 20 or more consecutive nucleotides, and the preceding sequences are merely exemplary. Transcripts of genes encoding proteins involved in light harvesting can therefore be targeted by the same RNAi or antisense molecule. A single RNAi or antisense molecule can also be designed to target only a transcript from a single gene encoding a protein involved in light harvesting by selecting a sequence that is unique to a single gene.

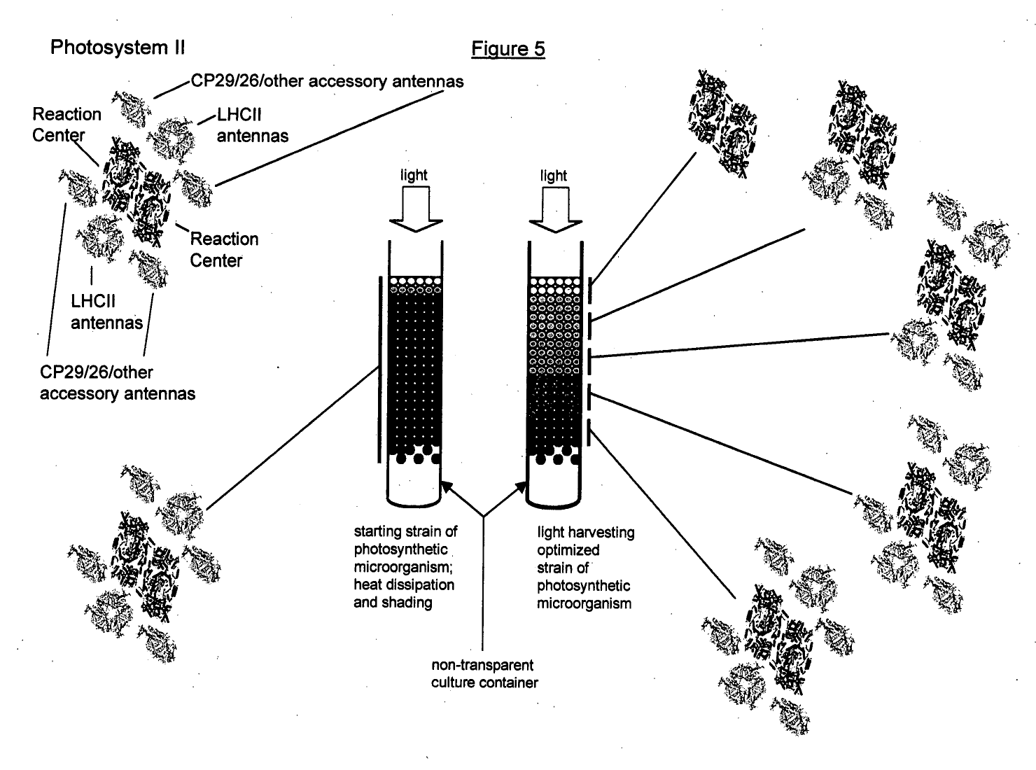

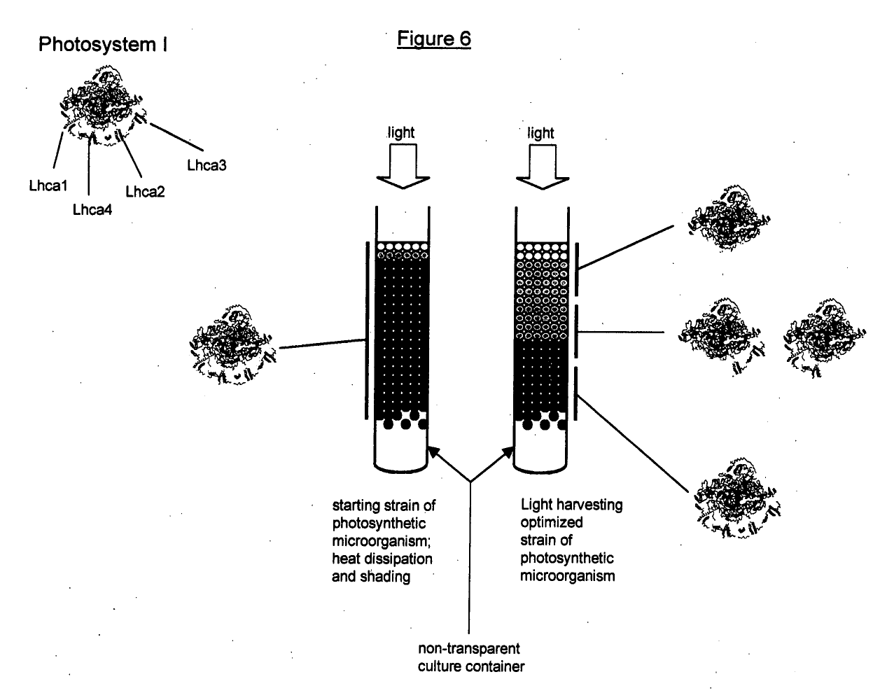

The expression of RNAi or antisense segments targeting antenna genes by light activated promoters causes, for example, the antenna expression pattern shown in FIGS. 5 and 6, where in a light harvesting optimized strain, the niunber of antennas expressed in a cell is dictated by the amount of light received by the cell. Cells in the top layers express the antisense or RNAi segment at a higher level than cells in the middle layers. Cells in the middle layers express the antisense or RNAi segment at a higher level than cells in the bottom layers. The variable expression level of the antisense or RNAi construct based on the position of a cell within a culture causes the population of cells in the culture to utilize light more efficiently than starting strains.

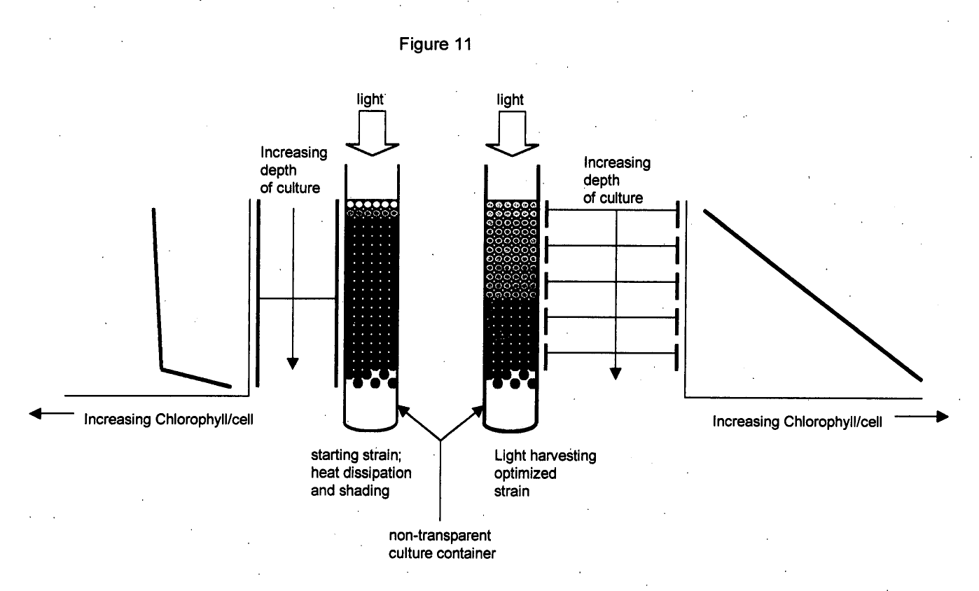

The expression of RNAi or antisense segments targeting chlorophyll biosynthesis genes by light activated promoters causes, for example, the antenna expression pattern shown in FIG. 11, where in a light harvesting optimized strain, the amount of chlorophyll in a cell is dictated by the amount of light received by the cell. Cells in the top layers express the antisense or RNAi segment at a higher level than cells in the middle layers. Cells in the middle layers express the antisense or RNAi segment at a higher level than cells in the bottom layers. The variable expression level of the antisense or RNAi construct based on the position of a cell within a culture causes the population of cells in the culture to utilize light more efficiently than starting strains.

ii. Codon Shifted Protein-Encoding Segments

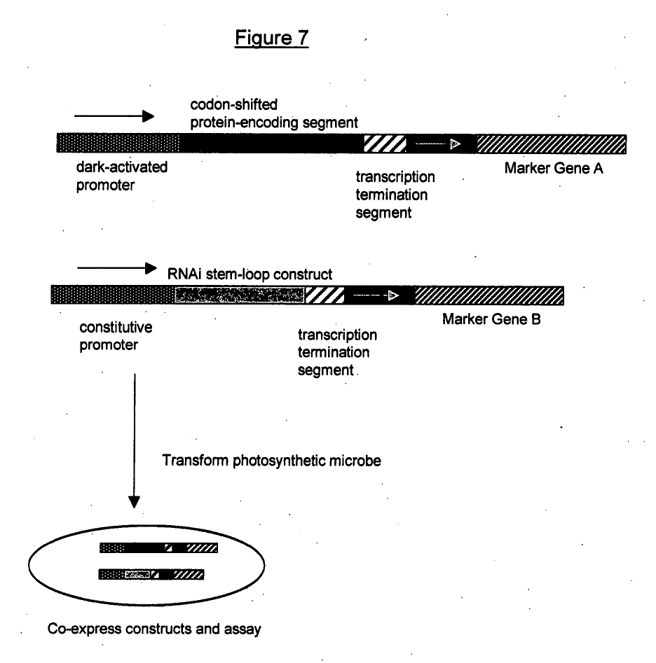

Codon shifted protein-encoding segments, which comprise cDNAs that encode proteins involved in light harvesting, can be expressed by heterologous promoters. These proteins are encoded by synthetic genes that differ in nucleotide sequence from the endogenous gene that encodes the protein in a wild type organism Specifically, these proteins are encoded by synthetic genes that utilize one or more codons that differ from the endogenous gene that encodes the protein in a wild type organism but encodes the same amino acid sequence. In other words, the synthetic gene encodes the same protein as an endogenous gene in an organism, but using one or more different codons to encode an amino acid. The codon shifted protein-encoding segment is expressed by a heterologous promoter, preferably a promoter that is activated by absence of light or a low (e.g.: 100 μmol photons/m⁻²/s⁻¹), but not high (e.g.:1000 μmol photons/m⁻²/s⁻¹) amount of light. An antisense or RNAi construct is coexpressed with the codon shifted protein-encoding segment, preferably from a constitutive promoter, and targets the transcript produced by the endogenous gene. The resulting cotransformed organism degrades the transcripts that are expressed by the endogenous gene encoding a protein involved in light harvesting, while the protein involved in light harvesting is expressed by the heterologous promoter. This coexpression design is depicted in FIG. 7.

The coexpression design described above and depicted in FIG. 7 causes, for example, the antenna expression pattern shown in FIGS. 5, 6, and 11 where in a light harvesting optimized strain, the number of antennas expressed in a cell is dictated by the amount of light received by a cell. Cells underneath the top layers express the codon shifted protein-encoding segment, at a level that correlates with the amount of light received by the cell. Cells that receive less light express the codon shifted protein-encoding segment at a higher level that cells that receive more light. All cells express one or more antisense or RNAi segments that degrade wild type antenna transcripts in all cells in the culture in a light-independent fashion. The variable expression level of the codon shifted protein-encoding segment based on the position of a cell within a culture causes the culture to utilize light more efficiently than non-transformed starting strains.

An alternative to using codon shifted protein encoding is to delete the targeted light harvesting gene from the genome of a photosynthetic microorganism and re-introduce the gene under the expression of a heterologous promoter. The heterologous promoter is preferably increasingly activated by decreasing levels of light, such as a dark activated promoter. Deleting or disrupting the endogenous gene from a photosynthetic microorganism achieves a similar effect as constitutively expressing an RNAi or antisense construct targeting transcripts produced from the endogenous gene.

iii. Other Proteins

Proteins that alter the function of proteins involved in light harvesting can also be expressed to cause alteration of light utilization. For example, monoclonal antibodies can be expressed in a photosynthetic microorganism to disrupt the function of certain proteins. For example, monoclonal antibodies to the tlal protein and enzymes involved in chlorophyll biosynthesis (such as hydroxymethylbilane synthase and glutamate-1-semialdehyde aminotransferase) can be expressed by light-activated promoters. Expression of such proteins disrupts normal photosynthetic fimction by interfering with signaling pathways and biosynthetic pathways necessary for normal light utilization efficiency. Methods for creation of monoclonal antibodies are known (see for example Shepherd, Monoclonal Antibodies: A Practical Approach, Oxford University Press 1999).

Genes that encode proteins that break down chlorophyll and antenna proteins can also be expressed by light activated promoters. Expression of such genes (such as MO25 and dee138 from Chlorella and nblA from Anabaena) from light activated promoters also causes cells in the top layer of a population of photosynthetic microorganisms to harvest less light than cells in the middle and bottom layers.

iv. Examples of Genes Encoding Proteins Involved in Light Harvesting for Design of Light Utilization Alteration Segments

Modulation of the presence and/or activity of proteins involved in light harvesting using light utilization alteration constructs is accomplished through altering the amount and/or type of various proteins in a photosynthetic microorganism. This is achieved through-expression of RNAi constructs, antisense constructs, codon shifted protein-encoding segments and other proteins as described above. The following genes and the proteins encoded by these genes are examples of candidates for modulation using light utilization alteration constructs.

TABLE 1
Examples of genes encoding proteins involved in light harvesting from various species of
photosynthetic microorganisms

| | | Gene | | | |
|---|---|---|---|---|---|
| | GenBank | Designation | | | |
| | Accession | or gene | | | |
| Gene | Number(s) | model* | Function | Species | Class |
| Lhcbm1 | 15430565 | | Antenna | Chlamydomonas | eukaryotic |
| | | | | reinhardtii | |
| Lhcbm2 | 15430563 | | Antenna | Chlamydomonas | eukaryotic |
| | | | | reinhardtii | |
| Lhcbm3 | 15430561 | | Antenna | Chlamydomonas | eukaryotic |
| | | | | reinhardtii | |
| Lhcbm4 | 4139215 | | Antenna | Chlamydomonas | eukaryotic |
| | | | | reinhardtii | |
| Lhcbm5 | 38234917 | | Antenna | Chlamydomonas | eukaryotic |
| | | | | reinhardtii | |
| Lhcbm6 | 167408 | | Antenna | Chlamydomonas | eukaryotic |
| | | | | reinhardtii | |
| Lhcbm8 | 12658405 | | Antenna | Chlamydomonas | eukaryotic |
| | | | | reinhardtii | |
| Lhcbm9 | 4139216 | | Antenna | Chlamydomonas | eukaryotic |
| | | | | reinhardtii | |
| Lhcbm11 | AF104630 | | Antenna | Chlamydomonas | eukaryotic |
| | | | | reinhardtii | |
| Lhca1 | | C_130138 | Antenna | Chlamydomonas | eukaryotic |
| | | | | reinhardtii | |
| Lhca2 | 27542568 | | Antenna | Chlamydomonas | eukaryotic |
| | | | | reinhardtii | |
| Lhca3 | | C_270001 | Antenna | Chlamydomonas | eukaryotic |
| | | | | reinhardtii | |
| Lhca4 | 4139222 | | Antenna | Chlamydomonas | eukaryotic |
| | | | | reinhardtii | |
| Lhca5 | | C_320083 | Antenna | Chlamydomonas | eukaryotic |
| | | | | reinhardtii | |
| Lhca6 | | C_1610027 | Antenna | Chlamydomonas | eukaryotic |
| | | | | reinhardtii | |
| Lhca7 | 19421770 | | Antenna | Chlamydomonas | eukaryotic |
| | | | | reinhardtii | |
| Lhca8 | | C_430022 | Antenna | Chlamydomonas | eukaryotic |
| | | | | reinhardtii | |
| Lhca9 | | Genie | Antenna | Chlamydomonas | eukaryotic |
| | | 218.10 | | reinhardtii | |
| Lhcb5 | 12060444 | | Antenna | Chlamydomonas | eukaryotic |
| | | | | reinhardtii | |
| Lhcb4 | 15430560 | | Antenna | Chlamydomonas | eukaryotic |
| | | | | reinhardtii | |
| Lhcq | | Genie | Antenna | Chlamydomonas | eukaryotic |
| | | 94.13 | | reinhardtii | |
| Ll818-1 | 1865772 | | Antenna | Chlamydomonas | eukaryotic |
| | | | | reinhardtii | |
| Ll818-2 | 1865770 | | Antenna | Chlamydomonas | eukaryotic |
| | | | | reinhardtii | |
| Elip1 | | Genie | Antenna | Chlamydomonas | eukaryotic |
| | | 814.2 | | reinhardtii | |
| Elip2 | | Genie | Antenna | Chlamydomonas | eukaryotic |
| | | 1248.0 | | reinhardtii | |
| Elip3 | | Genie | Antenna | Chlamydomonas | eukaryotic |
| | | 114.2 | | reinhardtii | |
| Elip4 | | C_570048 | Antenna | Chlamydomonas | eukaryotic |
| | | | | reinhardtii | |
| Elip5 | | Genewise | Antenna | Chlamydomonas | eukaryotic |
| | | 595.18.1 | | reinhardtii | |
| tla1 | AF534570 | | Signal | Chlamydomonas | eukaryotic |
| | AF534571 | | transduction | reinhardtii | |
| Magnesium chelatase | AF343974 | | Chlorophyll | Chlamydomonas | eukaryotic |
| | | | biosynthesis | reinhardtii | |
| Hydroxymethylbilane | BE725737 | | Chlorophyll | Chlamydomonas | eukaryotic |
| synthase | | | biosynthesis | reinhardtii | |
| Glutamate-1-semialdehyde | BF863318 | | Chlorophyll | Chlamydomonas | eukaryotic |
| aminotransferase | | | biosynthesis | reinhardtii | |
| NADPH: protochlorophyllide | BE352209 | | Chlorophyll | Chlamydomonas | eukaryotic |
| oxidoreductase | | | biosynthesis | reinhardtii | |
| protochlorophyllide | U36752 | | Chlorophyll | Chlamydomonas | eukaryotic |
| oxidoreductase | | | biosynthesis | reinhardtii | |
| protochlorophyllide | X60490 | | Chlorophyll | Chlamydomonas | eukaryotic |
| reductase | | | biosynthesis | reinhardtii | |
| L1818 | P22686 | | Antenna | Chlamydomonas | eukaryotic |
| | | | | moewusii | |
| L1818 | Q03965 | | Antenna | Chlamydomonas | eukaryotic |
| | | | | eugamentos | |
| Lhcbm | AAT66413 | | Antenna | Chlorella pyrenoidosa | eukaryotic |
| MO25 | AJ238632 | | Chlorophyll | Chlorella | eukaryotic |
| | | | breakdown | protothecoides | |
| dee138 | AJ238630 | | Chlorophyll | Chlorella | eukaryotic |
| | | | breakdown | protothecoides | |
| dee8 | AJ238625 | | Chlorophyll | Chlorella | eukaryotic |
| | | | breakdown | protothecoides | |
| CP-47 | AB001684 | | Antenna | Chlorella vulgaris | eukaryotic |
| Magnesium chelatase | NP_045914 | | Chlorophyll | Chlorella vulgaris | eukaryotic |
| | | | biosynthesis | | |
| protochlorophyllide | AB001684 | | Chlorophyll | Chlorella vulgaris | eukaryotic |
| reductase ChlB subunit | | | biosynthesis | | |
| fucoxanthin-chlorophyll a/c | U66185 | | Antenna | Phaeodactylum | eukaryotic |
| light-harvesting protein | | | | tricornutum | |
| fucoxanthin-chlorophyll | X55157 | | Antenna | Phaeodactylum | eukaryotic |
| protein 3 | | | | tricornutum | |
| fucoxanthin chlorophyll | X55156 | | Antenna | Phaeodactylum | eukaryotic |
| protein 2 | | | | tricornutum | |
| fucoxanthin chlorophyll | X55250 | | Antenna | Phaeodactylum | eukaryotic |
| protein 1 | | | | tricornutum | |
| light harvesting protein | Z24768 | | Antenna | Phaeodactylum | eukaryotic |
| | | | | tricornutum | |
| Lhca | AAD55568 | | Antenna | Volvox carteri | eukaryotic |
| Lhca | AAD55569 | | Antenna | Volvox carteri | eukaryotic |
| Lhca | S72223 | | Antenna | Volvox carteri | eukaryotic |
| Lhca | AAB40979 | | Antenna | Volvox carteri | eukaryotic |
| L1818 | AAD55567 | | Antenna | Volvox carteri | eukaryotic |
| Delta-aminolevulinic acid | CAC36225 | | Chlorophyll | Volvox carteri | eukaryotic |
| dehydratase | | | biosynthesis | | |
| fucoxanthin chlorophyll a/c | AJ002017 | | Antenna | Thalassiosira | eukaryotic |
| binding protein | | | | weissflogii | |
| iron stress-induced | AP005372 | | Antenna | Thermosynechococcus | prokaryotic |
| chlorophyll-binding protein | | | | elongatus | |
| light-harvesting protein | AP005369 | | Antenna | Thermosynechococcus | prokaryotic |
| | | | | elongatus | |
| phycobilisome core | NC_004113 | | Antenna | Thermosynechococcus | prokaryotic |
| component | | | | elongatus BP-1 | |
| Magnesium chelatase | NP_682301 | | Chlorophyll | Thermosynechococcus | prokaryotic |
| | | | biosynthesis | elongatus BP-1 | |
| CP47 homolog | AE017162 | | Antenna | Prochlorococcus | prokaryotic |
| | | | | marinus subsp. | |
| | | | | marinus str. | |
| | | | | CCMP1375 | |
| Magnesium protoporphyrin | L47126 | | Chlorophyll | Synechocystis sp. | prokaryotic |
| IX methyl transferase | | | biosynthesis | PCC 6803 | |
| phycoerythrin alpha | AF169367 | | Antenna | Synechocystis sp. | prokaryotic |
| subunit | | | | BO8402 | |
| phycoerythrin beta subunit | AF169367 | | Antenna | Synechocystis sp. | prokaryotic |
| | | | | BO8402 | |
| Phycobilisome core protein | NC_000911 | | Antenna | Synechocystis sp. | prokaryotic |
| | | | | PCC 6803 | |
| phycoerythrin alpha | AF304135 | | Antenna | Prochlorococcus | prokaryotic |
| subunit | | | | marinus MIT9303 | |
| nblA | AJ504665 | | Phycobilisome | Anabaena variabilis | prokaryotic |
| | | | degradation | | |
| chlorophyll synthase | AP003596 | | Chlorophyll | Anabaena PCC7120 | prokaryotic |
| | | | biosynthesis | | |
| allophycocyanin alpha | U96137 | | Antenna | Anabaena PCC7120 | prokaryotic |
| subunit | | | | | |
| allophycocyanin beta | U96137 | | Antenna | Anabaena PCC7120 | prokaryotic |
| subunit | | | | | |
| Allophycocyanin beta-18 | BX569692 | | Antenna | Synechococcus sp. | prokaryotic |
| subunit | | | | WH8102 | |
| CP47 | BX569694 | | Antenna | Synechococcus sp. | prokaryotic |
| | | | | WH8102 | |
| CP43 | NC_005070 | | Antenna | Synechococcus sp. | prokaryotic |
| | | | | WH 8102 | |
| chlorophyll synthase | NC_005070 | | Chlorophyll | Synechococcus sp. | prokaryotic |
| | | | biosynthesis | WH 8102 | |
| NADPH: protochlorophyllide | U30252 | | Chlorophyll | Synechococcus sp. | prokaryotic |
| oxidoreductase | | | biosynthesis | PCC 7942 | |

*from C. reinhardtii genome

a Photosystem I Antenna Genes

PSI has four antenna proteins that surround the core complex in a semicircle-shaped ring. (see FIG. 5 and Nature 2003 Dec. 11;426(6967):630-5). The antenna proteins bind chlorophyll and other pigments. These antenna proteins evolved from a common ancestor gene and have a high level of amino acid sequence identity. Although only four proteins can surround a PSI core complex, there are at least nine genes that encode PSI antenna subunit proteins in the green algae Chlamydomonas reinhardtii. In Chlamydomonas reinhardtii, these proteins are referred to as Lhca1, Lhca2, Lhca3, Lhca4, Lhca5, Lhca6, Lhca7, Lhca8 and Lhca9, listed in Table 1 (see Curr Genet. 2004 February; 45(2):61-75 for nomenclature). PSI antenna genes from other species are known, such as genes from Volvox carteri (GenBank Accession numbers AAD55568, AAD55569, S72223 and AAB40979).

b. Photosystem II Antenna Genes

The PSII complex comprises trimers of light harvesting antennas, referred to as LCHII, associated with it. In C. reinhardtii, these proteins are referred to as Lhcbm1, Lhcbm2, Lhcbm3, Lhcbm4, Lhcbm5, Lhcbm6, Lhcbm8, Lhcbm9 and Lhcbm11, listed in Table 1. (see Curr Genet. 2004 February; 45(2):61-75 for nomenclature). In addition, single light harvesting proteins known as “CP” proteins are also associated with the complex (Biochemistry 2003, 42, 608-613; Nature 2004 Mar. 18;428(6980):287-92). In C. reinhardtii, these proteins are referred to as Lhcb4 and Lhcb5 (see Curr Genet. 2004 February; 45(2):61-75). The molecular weight of these proteins varies between photosynthetic organisms. The light harvesting proteins of PSII bind chlorophyll and other pigments. PSII antenna genes from numerous species are known, such as genes from Chlorella pyrenoidosa (GenBank Accession number AAT66413) and Volvox carteri (GenBank Accession number AAD55567).

c. Chlorophyll Biosynthesis Genes

Genes that encode proteins that participate in the biosynthesis of chlorophyll are candidates for modulation by light utilization alteration constructs. Examples of such genes and proteins are:

Hydroxymethylbilane synthase (GenBank Accession number BE725737 (Chlamydomonas reinhardtii));

Glutamate-1-semialdehyde aminotransferase, (GenBank Accession numbers U03632 and U03633 (Chlamydomonas reinhardtii); S13326 (Synechococcus sp. PCC 6301), AAP79194 (Bigelowiella natans));

NADPH:protochlorophyllide oxidoreductase (GenBank Accession number U36752 (Chlamydomonas reinhardtii));

Magnesium chelatase (GenBank Accession numbers AF343974 (Chiamydomonas reinhardtii); NP_{—}045914 (Chlorella vulgaris); NP_{—}050837 (Nephroselmis olivacea), NP_{—}682301 (Thermosynechococcus elongatus BP-1); NP_{—}484196 (Anabaena sp. strain PCC 7120); ZP_{—}00326592 (Trichodesmium erythraeum IMS101));

Delta-aminolevulinic acid dehydratase (GenBank Accession numbers U19876 (Chlamydomonas reinhardtii); CAC36225 (Volvox carteri);

Chlorophyll b synthase (GenBank Accession number BAA82481 (Dunaliella salina));

Chlorophyll a oxygenase (GenBank Accession number BAA33964 (Chlamydomonas reinhardtii).

d. Other Genes Encoding Proteins Involved in Light Harvesting

Other genes not mentioned above that are involved in light harvesting are also candidates for modulation by light utilization alteration constructs. An example of such a gene is tlal (GenBank Accession numbers AF534570 and AF534571 (Chlamydomonas reinhardtii), which regulates chlorophyll content of cells through intracellular signaling pathways. In addition, the Elip1, Elip2, Elip3, Elip4, Elip5 and LI818r-1 and LI818r-3 proteins from C. reinhardtii are also candidates for modulation by light utilization alteration constructs (see Curr Genet. 2004 February; 45(2):61-75). GenBank accession numbers for examples of genes of the LI818 class are T08175 (Chlamydomonas reinhardtii); P22686 (Chlamydomonas moewusii); Q03965 (Chiamydomonas eugamentos). GenBank accession numbers for examples of genes of the Elip class are are C_{—}570048 (Chlamydomonas reinhardtii) and P27516 (Dunaliella bardawil).

Additional genes encoding proteins involved in light harvesting are listed in Table 1. Genetic constructs and methods of the invention include light utilization alteration segments and uses thereof that comprise genes encoding proteins involved in light harvesting from all photosynthetic microorganisms, both eukaryotic and prokaryotic.

More genes encoding proteins involved in light harvesting can be found in known genome sequences such as those available at http://genomejgi-psf.org/finished_microorganisms. Fully sequenced genomes of prokaryotic and eukaiyotic photosynthetic microorganisms include Anabaena variabilis ATCC 29413, Chloroflexus aurantiacus, Nostocpunctiforme, Rhodobacter sphaeroides, Synechococcus elongatus PCC 7942, Synechococcus sp. strain WH8102, Rhodopseudomonas palustris, Prochlorococcus marinus MIT9313, Prochlorococcus marinus MED4 and Chiamydomonas reinhardtii.

Other genes encoding proteins involved in light harvesting encode proteins that have at least 40%, 50%, 60%, 70%, 80%, 90%, 95%, and 98% amino acid identity with the proteins cited herein.

### C. Transcriptional Termination Segment

It is preferred that a light utilization alteration segment be in operable linkage with a transcriptional termination segment. Exemplary transcriptional termination segments are SEQ ID NOs: 28, 49 and 58. Many different transcriptional termination segments can be used in light utilization alteration segments. Such segments are not strictly necessary to perform methods of the invention but they are preferred.

### D. Promoters in Operable Linkage with a Light Utilization Alteration Segment

Any promoter, naturally occurring or synthetic, including sections of naturally occurring promoters, can be placed in operable linkage with a light utilization alteration segment. Constitutive promoters as well as promoters that are activated by a stimulus can be placed in operable linkage with a light utilization alteration segment. In a preferred embodiment, a stimulus that activates a promoter in operable linkage with a light utilization alteration segment is light. It is also preferred that a promoter used to drive a light utilization alteration segment is active in relatively high levels of CO₂ compared to atmospheric air, such as 1-10%, more preferably 2-6%, more preferably 3-5%. It is also preferred that a light-activated promoter exhibit an increasing level of activity in response to increasing levels of light.

Sections of a promoter sufficient to confer light activated transcription can be placed in operable linkage with a light utilization alteration segment. For example, the −255 to −1 section (with respect to start of translation) of the C. reinhardtii lhcbm1 gene can be placed in operable linkage with a light utilization alteration segment and expressed in C. reinhardtii (lhcbm1 promoter sequence analyzed in Hahn, Curr Genet (1999) January; 34(6):459-66). In a library of light utilization alteration constructs, different sections of a plurality of light activated promoters can be placed in operable linkage with one or more light utilization alteration segments. For example, sections corresponding to the −1500 to −1, −1000 to −1, −500 to −1, and −250 to −1 (with respect to start of translation) sections of a plurality of promoters can be placed in operable linkage with one or more light utilization alteration segments. The 3′ end of a promoter can also be farther upstream than −1 with respect to start of translation. Transcription usually initiates approximately 20-30 base pairs downstream of a TATA box in a promoter.

In one embodiment, a plurality of staggered fragments are amplified from each promoter of a plurality of genes that are activated by light. The plurality of fragments corresponds to different 5′ and 3′ boundaries within the promoter region. It is preferred but not required that a sense primer for amplification of a promoter fragment anneal upstream of the TATA box of a promoter, and that an opposing primer anneal downstream of the start site for transcription. Amplification of multiple fragments of a light activated promoter allows for a functional sampling of different strengths of light activation by the fragments when they are cloned into operable linkage with a light utilization alteration segment.

Exemplary light-activated genes in C. reinhardtii are listed in Table 2. Sections of the promoters of these genes can be amplified by PCR and incorporated into light utilization alteration constructs using the C. reinhardtii genome sequence to design primers for amplification. Preferred promoters are activated in high light (such as 1000 μmol photon m⁻² s⁻¹) and high CO₂ (such as 4%). Additional examples of light-activated C. reinhardtii genes can be found in Photosynthesis Research 75: 111-125, 2003.

TABLE 2
Examples of Light activated C. reinhardtii genes

| GenBank Accession Number | Gene Name or Description | Additional Acc. No.(s) |
|---|---|---|
| 894005B12.x2 | Similar to Arabidopsis Lil3 protein | |
| 894093F09 | Copper response target 1 protein | (AF337038) |
| 894081G12 | Superoxide dismutase (Fe) | (U22416) BE725229 |
| 894012D09 | Geranylgeranyl hydrogenase | BE121489 |
| 894086H03 | Sterol-C-methyltransferase | BE725843 |
| 963038E06 | Compare (U13167) YptC4, small G-proteins | BF862816 |
| 894097E05 | Chlorophyll a/b-binding protein Ll818r-3 | (X95326) BE761255 |
| 894086C03 | Hydroxymethylbilane synthase | BE725737 |
| 963042G07 | Glutamate-1-semialdehyde aminotransferase | (U03632) BF863318 |
| 894057D06 | NADPH: protochlorophyllide oxidoreductase | (U36752) BE352209 |
| 894013H01 | Unknown | BE121633 |
| 963069C08.x1 | Similar to Arabidopsis | AC079284_5 |
| 894013A09 | Similar to an unidentified Volvox protein | BE121543 |
| 963029F06 | Similar to Arabidopsis | AL138642 BF861885 |
| 963047D02 | S-adenosyl methionine synthetase; | (AF008568) |
| BF863876 | Unknown | |
| 894040F03 | Phosphoglycerate kinase | (U14912) BE238167 |
| 894004C06 | Magnesium chelatase ChlI subunit | (AF343974) |
| BE024621 | Unknown | |
| 894052A01 | LHC-blastx similar to CAB protein CP26 | (AB050007) |
| BE351814 | Unknown | |
| 963042A01 | Lhca4 | BF863200 |
| 963041 | OEE3 | (X13832) BF863140 |
| 894069D01 | Sulfite reductase | BE453250 |
| 894021A12 | Ribose 5-phosphate isomerase | BE129029 |
| 963028B11 | Similar to bacterial D-3-phosphoglycerate dehyd. | BF861822 |
| 894029D02 | Ornithine decarboxylase | BE212342 |
| 894001G07 | Delta 9 desaturase | BE024254 1.5 |
| 963047D08.x1 | Ubiquinol-cytochrome-c reductase | BM519228 |
| 894038C12 | Glutamine synthetase GS1 (cytosolic) | BE237804 |
| 894066E11 | Copper response defect 1 protein; | (AF237671) |
| BE452896 | Unknown | |
| 894001E02 | PRT1, translation initiation factor 3 (eIF3) | BE024207 |
| 894010B12 | Serine hydroxymethyltransferase | BE056562 |
| 894020F09 | Phosphoglucomutase | BE128972 |
| 894049E03.x1 | Unknown | |
| 894068F02 | Unknown | BE453128 |
| 894002G05 | Unknown | BE024406 |
| 894077H12 | Similar to Arabidopsis MKP11.2 | BE724672 |
| 963038F01.x2 | Similar to Arabidopsis T13D8.29 | BF862826 |
| 894044G05 | Similar to Porphyra ORF99 (NC_000925) | BE337246 |
| 894026D10 | Unknown | BE211827 |
| 894066H09 | Unknown | BE452950 |
| 894057G07 | Unknown | BE352237 |
| 963024C04.x1 | Unknown | BM519021 |
| 894102E09.x1 | Unknown | BM518913 |
| 963029C09 | Unknown | BF861863 |
| 894014D02 | Unknown | BE121701 |
| 894099H02 | Unknown | BE761521 |
| 894065G04 | Unknown | BE452774 |
| 963046H09 | Similar to Volvox sulfated surface glycoprotein 185 | BF863819 |

Promoters from the above genes can be isolated as follows, using the exemplary method disclosed below for amplifying sections of the C. reinhardtii magnesium chelatase CHI subunit gene promoter. The Genbank accession number AF343974, designating the magnesium chelatase ChlI subunit gene, can be used to identify the cDNA sequence of the magnesium chelatase CHlI subunit gene search under the “nucleotide” function of the National Center for Biotechnology Information at http://www.ncbi.nlm.nih-gov. A region of nucleotides of the cDNA sequence, preferably at least 25 contiguous nucleotides and more preferably at least 50 contiguous nucleotides, are then used to search the C. reinhardtii nuclear genome at the Chlamy EST database at http://www.biology.duke.edu/chlamy_genome/blast/blast_form.html. 100% identical sequences identified from the Chlamy EST database correspond to the genomic sequence of the magnesium chelatase ChlI subunit gene. These sequences identify the exact position of the magnesium chelatase ChlI subunit gene within the C. reinhardiii genome, which is in scaffold 9 of the genome sequence at approximately base pair 625,800. This information is used to navigate through scaffold 9 of the genome in the “browse” function of the C. reinhardtii genome at hftp://genome.jgi-psf.org/chlre2/chlre2.home.html to locate the genomic sequence of the magnesium chelatase ChlI subunit gene. Navigation in the browser to the region surrounding position 625,000 shows the genomic structure of the magnesium chelatase ChlI subunit gene spanning approximately positions 622,200 to 626,400. Clicking on the structure of the gene pulls up the annotated page describing the magnesium chelatase ChlI subunit gene (identified as C_{—}90171). Clicking on the structure of the gene pulls up the genomic region of the gene, with 3′ and 5′ untranslated sections of the cDNA designated in blue, exons designated in red, and introns and upstream sequence designated in black Adjusting the “upstream/downstream padding” number alters the amount of upstream and downstream sequence displayed. The exact start site of transcription is not always known, however transcription must initiate by at least the base pair immediately upstream of the start codon of any gene.

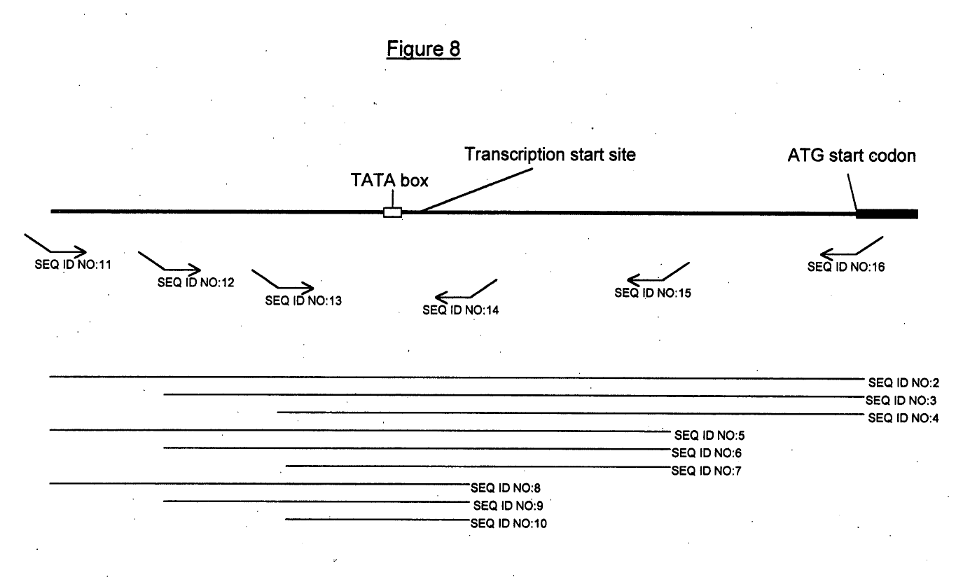

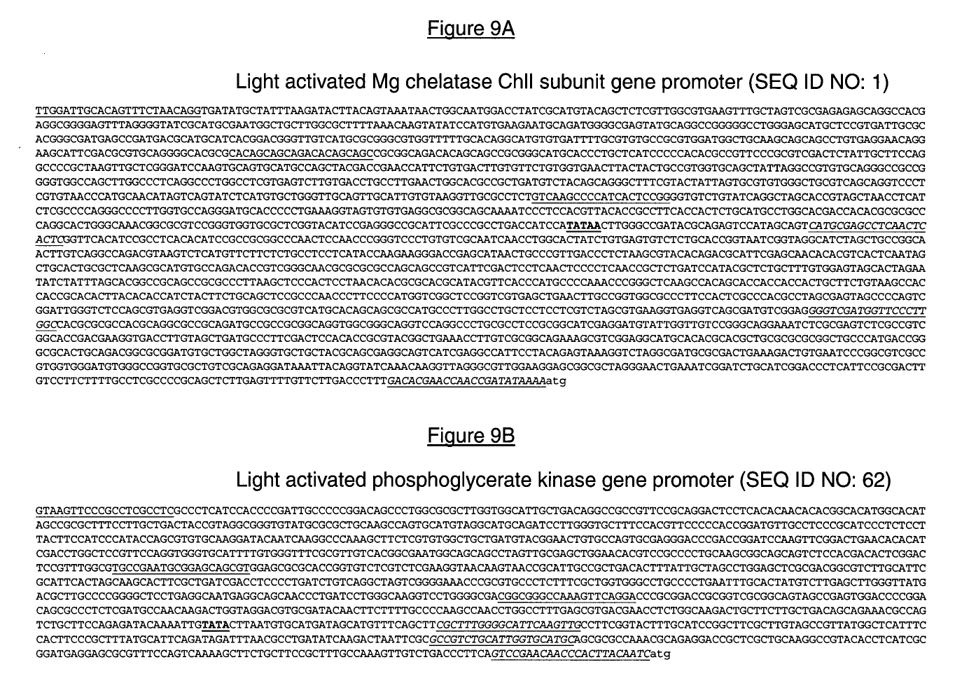

Promoter sequences can be generated through amplification using genomic DNA sequence of a photosynthetic microorganism as a template. The genomic DNA sequence can be isolated genomic DNA, cloned genomic fragments such as bacterial artificial chromosomes, amplified genomic fragments, and other sources. For example, FIGS. 8 and 9A depict amplification of various sizes of promoter fragments from the upstream region of the magnesium chelatase ChlI subunit gene (SEQ ID NO:1). A TATA box is located at approximately −1414 with respect to initiation of translation. A series of nine staggered promoter sections (SEQ ID NOs: 2-10) can be isolated by amplification of C. reinhardtii genomic DNA using primers of SEQ ID NOs: 11-16, as depicted in FIG. 8 and listed in Table 3.

TABLE 3
Mg chelatase promoter fragments and primers for amplification

| | | | | | Fragment |
|---|---|---|---|---|---|
| | | | | | length |
| Promoter | | Antisense | Sense Linker | Antisense | including |
| Section | Sense primer | primer | Tail | Linker Tail | linker tails |
| SEQ ID NO: 2 | SEQ ID NO: 11 | SEQ ID NO: 16 | SEQ ID NO: 17 | SEQ ID NO: 18 | 2579 bp |
| SEQ ID NO: 3 | SEQ ID NO: 12 | SEQ ID NO: 16 | SEQ ID NO: 17 | SEQ ID NO: 18 | 2164 bp |
| SEQ ID NO: 4 | SEQ ID NO: 13 | SEQ ID NO: 16 | SEQ ID NO: 17 | SEQ ID NO: 18 | 1733 bp |
| SEQ ID NO: 5 | SEQ ID NO: 11 | SEQ ID NO: 15 | SEQ ID NO: 17 | SEQ ID NO: 18 | 1992 bp |
| SEQ ID NO: 6 | SEQ ID NO: 12 | SEQ ID NO: 15 | SEQ ID NO: 17 | SEQ ID NO: 18 | 1577 bp |
| SEQ ID NO: 7 | SEQ ID NO: 13 | SEQ ID NO: 15 | SEQ ID NO: 17 | SEQ ID NO: 18 | 1146 bp |
| SEQ ID NO: 8 | SEQ ID NO: 11 | SEQ ID NO: 14 | SEQ ID NO: 17 | SEQ ID NO: 18 | 1219 bp |
| SEQ ID NO: 9 | SEQ ID NO: 12 | SEQ ID NO: 14 | SEQ ID NO: 17 | SEQ ID NO: 18 | 804 bp |
| SEQ ID NO: 10 | SEQ ID NO: 13 | SEQ ID NO: 14 | SEQ ID NO: 17 | SEQ ID NO: 18 | 373 bp |

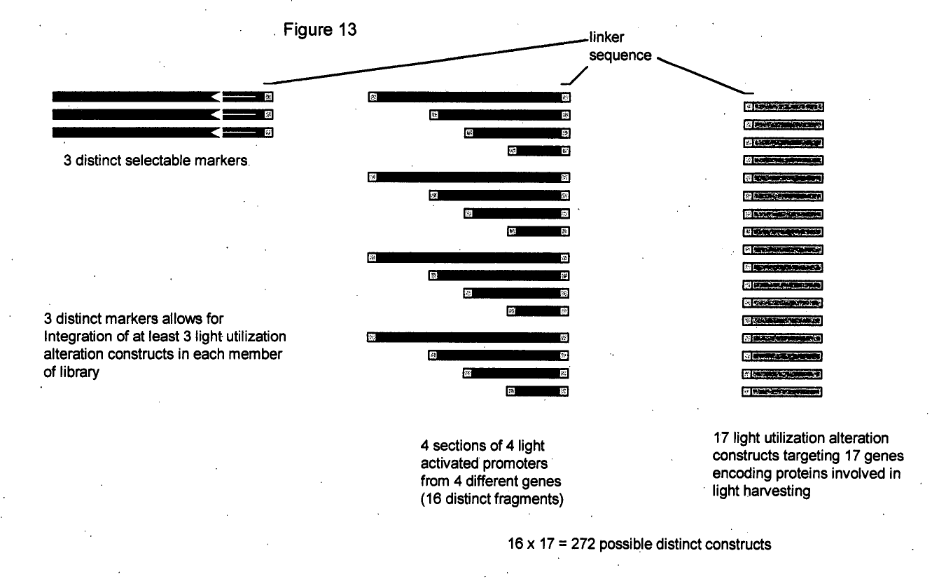

The design of the PCR primers used to amplify these promoter fragments (SEQ ID NOs: 11-16) includes linker tails on the 5′ ends of the sense and antisense oligonucleotides. These 27 nucleotide linker tail sequences (SEQ ID NOs: 17-18) are annealing partners for other fragments described in later sections, allowing the combinatorial construction of light utilization alteration constructs through annealing of complementary linker sequences followed by extension by a polymerase as shown in FIG. 13.

The above process can be performed for generation of a library of different promoter strengths in response to one or more stimuli, including nutrient deprivation, addition of a compound or ion to the culture media, light of a particular wavelength, and other stimuli. Knowledge of the stimuli that activate a promoter is not necessary to generate such a library of promoter fragments.

Promoter sequences from any gene, including light activated genes, can amplified using PCR, including the promoters of the light activated genes listed in table 1 of Photosynthesis Research 75: 111-125, 2003. Other light-activated promoters are also known in C. reinhardtii (Mol Gen Genet. 1995 Oct. 25;248(6):727-34; Plant Mol Biol. 1998 April; 36(6):929-34), including promoters activated by specific wavelength ranges (Plant Physiol. 1995 October; 109(2):471-479). Methods of PCR are known in the art (see for example PCR: A Practical Approach M. J. McPherson, P. Quirke, G. R. Taylor, Oxford University Press (February 1992) ISBN 0199631964; Molecular Cloning: A Laboratory Manual, Sambrook et al. (3d edition, 2001, Cold Spring Harbor Press; and U.S. Pat. No. 4,683,202). Error prone PCR can also be used to generate variability in amplification products (Technique (1989) 1, 11-15).

Light-activated promoters have been identified from numerous species of photosynthetic microorganisms. Examples of light-activated promoters from C. reinhardtii include those described in: (Hahn, Curr Genet (1999) January; 34(6):459-66; Loppes, Plant Mol Biol 2001 January; 45(2):215-27; Villand, Biochem J 1997 Oct. 1;327 (Pt 1):51-7; Muller, Gene (1992) Feb. 15;111(2):165-73; von Gromoff, Mol Cell Biol (1989) Sep. 9(9):3911-8; Mol Cell Biol Res Commun. 2000 May 3(5):292-8; Mol Cell Biol. 1992 Nov. 12(11):5268-79). C. reinhardtii promoter sequences that allow expression only in the dark are also known (Proc Natl Acad Sci U S A 1993 Feb. 15;90(4):1556-60).

Promoters from Chlorella viruses can be incorporated into light utilization alteration constructs for expression in Chlorella (see Virology, 2004 Aug. 15;326(1):150-9; Virology, 2004 Jan. 5;318(1):214-23). Promoters from Volvox can also be incorporated into a light utilization alteration construct (see Proc Natl Acad Sci U S A. 1996 Jan. 23;93(2):669-73), and discrete promoter elements and enhancers that activate Volvox transcription are also known (Curr Genet. 1995 Sep. 28(4):333-45; Gene. 1995 Jul. 4;160(1):47-54; Genes Dev. 2001 Jun. 1;15(11):1449-60). Promoters active in Phaeodactylum tricornutum and Thalassiosira weissflogii can also be incorporated into a light utilization alteration construct (Falciatore A, Casotti R, Leblanc C, Abrescia C, Bowler C, PMID, 10383998, 1999 May 1(3):239-251 (Laboratory of Molecular Plant Biology, Stazione Zoologica, Villa Comunale, I-80121 Naples, Italy)). It has also been demonstrated that promoters from one species of microalgae can be functional when placed in operable linkage with a gene and transformed into an organism of a different species, such as the activity of C. reinhardtii promoters in Chlorella (see Mar Biotechnol (NY). 2002 Jan. 4(1):63-73) and the activity of Chlorella promoters in organisms such as Arabidopsis, potato plants, maize, Sorghum, E. cohi, Erwinia, Pseudomonas, and Xanthomonas bacteria (Biochem Biophys Res Commun. 1994 Oct. 14;204(1):187-94). Promoters from algal species are frequently active in organisms from other species. Other light activated promoter systems can be used in a plurality of species (see Shimizu-Sato, Nat Biotechnol 2002 Oct. 20(10):1041-4).

Light and dark-activated promoters and other light and dark responsive regulatory elements are known in prokaryotic photosynthetic microorganisms: Synechococcus (see FEMS Microbiol Lett. 2004 Jun. 15;235(2):341-7; Mol Microbiol. 2004 May; 52(3):837-45; Plant Cell Physiol. 1999 April; 40(4):448-52); Fremyella diplosiphon (see J Mol Biol. 1988 Feb. 5;199(3):447-65; J Bacteriol. 1994 October; 176(20):6362-74; J Bacteriol. 1993 March; 175(6):1806-13; J Bacteriol. 1994 October; 176(20):6362-74);Anabaena (see EMBO J. 1987 Apr. 6(4):871-84); Synechocystis (see FEBS Lett. 2003 Nov. 20;554(3):357-62; Mol Microbiol. 2003 August; 49(4):1019-29; Mol Cell Biol Res Commun. 2000 May 3(5):292-8); Mol Microbiol. 1994 Jun. 12(6):1005-12).

While most of the aforementioned promoters are endogenous to the species listed, some light-activated promoters in higher plants have been shown to function in a light regulated fashion in cyanobacteria (see Plant Cell Physiol. 1999 April; 40(4):448-52).

Promoters and sections of promoters can be used to drive light utilization alteration segments. In addition, sections of different promoters, as well as individual response elements from different promoters, can be incorporated into promoter segments. Different sections of promoters can also be attached to form a library of promoter sections.

### E. Marker Component

Light utilization alteration constructs contain a screenable or selectable marker component. When a single light utilization alteration construct or a plurality of constructs (such as a library as described in example 1) are used to transform photosynthetic microorganisms, inclusion of a screenable or selectable marker enables the isolation of independent strains that have had one or more light utilization alteration constructs incorporated into a genome. In the case of a eukaryotic photosynthetic microbe, a light utilization alteration construct can be integrated into the chloroplast, nuclear, or mitochondrial genome.

Many selectable markers are known that can be used in photosynthetic microorganisms. For example, selectable markers for use in Chlamydomonas are known, including but not limited to markers imparting spectinomycin resistance (Mol Cell Biol (1999) Oct. 19(10):6980-90), kanamycin and amikacin resistance (Mol Gen Genet (2000) April; 263(3):404-10), zeomycin and phleomycin resistance (Mol Gen Genet (1996) Apr. 24;251(1):23-30), and paromycin and neomycin resistance (Gene (2001) Oct. 17;277(1-2):221-9). Screenable markers are available in Chlamydomonas, such as the green fluorescent protein (Plant J (1999) Aug. 19(3):353-61) and the Renilla luciferase gene (Mol Gen Genet (1999) October; 262(3):421-5.

Selectable markers for use in other eukaryotic photosynthetic microorganisms are also known (see for example Curr Microbiol. 1997 December; 35(6):356-62 (Chlorella vulgaris); Mar Biotechnol (NY). 2002 Jan. 4(1):63-73 (Chlorella ellipsoidea); Mol Gen Genet. 1996 Oct. 16;252(5):572-9 (Phaeodactylum tricornutum); Plant Mol Biol. 1996 April; 31(1):1-12 (Volvox carteri); Proc Natl Acad Sci U S A. 1994 Nov. 22;91(24):11562-6 (Volvox carteri); (Falciatore A, Casotti R, Leblanc C, Abrescia C, Bowler C, PMID: 10383998, 1999 May 1(3):239-251 (Laboratory of Molecular Plant Biology, Stazione Zoologica, Villa Comunale, I-80121 Naples, Italy) (Phaeodactylum tricornutum and Thalassiosira weissflogii).

Selectable markers for use in prokaryotic photosynthetic microorganisms are known in the art (Koksharova, Appl Microbiol Biotechnol 2002 February; 58(2):123-37 (various species); Mol Genet Genomics. 2004 February; 271(1):50-9 (Thermosynechococcus elongates); Plant Physiol. 1995 March; 107(3):703-708, Proc Natl Acad Sci U S A. 2002 Mar. 19;99(6):4109-14 (Sechococcus PCC 7942); Mar Pollut Bull. 2002;45(1-12):163-7 (Anabaena PCC 7120); Proc Natl Acad Sci U S A. 1984 March; 81(5):1561-5 (Anabaena (various strains)); Proc Natl Acad Sci U S A. 2001 Mar. 27;98(7):4243-8 (Synechocystis); Wirth, Mol Gen Genet 1989 March; 216(1):175-7 (various species)).

Fluorescent proteins for use as screenable markers are also available for expression in prokaryotic photosynthetic microorganisms (Mol Microbiol. 2003 June; 48(6):1481-9; (Synechocystis); J Bacteriol. 2002 May; 184(9):2491-9 Oabaena)).

Screenable or selectable markers are placed in operable linkage with promoter. Marker genes are preferably in operable linkage with a constitutive promoter.

### III Construction of Libraries

### A. Starting Strain

Photosynthetic microorganisms are transformed with light utilization alteration constructs. The strain of photosynthetic microorganism is referred to herein as a starting strain Starting strains can be prokaryotic or eukaryotic. A starting strain can be a wild-type strain of a photosynthetic microorganism, or a strain that has been genetically transformed.

TABLE 4
Exemplary Starting Strains

| | Strain | |
|---|---|---|
| | Accession | |
| Species | Number^{†} | Class |
| Volvox carteri | UTEX 1877 | eukaryotic |
| Volvox capensis | UTEX 2712 | eukaryotic |
| Volvox carteri | UTEX 2170 | eukaryotic |
| Volvox gigas | UTEX 1895 | eukaryotic |
| Phaeodactylum tricornutum | UTEX 640 | eukaryotic |
| Phaeodactylum tricornutum | UTEX 2089 | eukaryotic |
| Phaeodactylum tricornutum | UTEX 2090 | eukaryotic |
| Chlorella vulgaris | UTEX 30 | eukaryotic |
| Chlorella vulgaris | UTEX 1811 | eukaryotic |
| Chlorella fusca | UTEX 343 | eukaryotic |
| Chlorella fusca | UTEX 1801 | eukaryotic |
| Chlorella kessleri | UTEX 2228 | eukaryotic |
| Chlamydomonas reinhardtii | UTEX 90 | eukaryotic |
| Chlamydomonas reinhardtii | UTEX 90 | eukaryotic |
| Chlamydomonas reinhardtii | CC-124 | eukaryotic |
| Chlamydomonas reinhardtii | CC-125 | eukaryotic |
| Chlamydomonas moewusii | UTEX 2018 | eukaryotic |
| Chlamydomonas eugamentos | UTEX 4 | eukaryotic |
| Anabaena variabilis | UTEX B 377 | prokaryotic |
| Anabaena verrucosa | UTEX 1619 | prokaryotic |
| Anabaena variabilis | ATCC 29413 | prokaryotic |
| Anabaena affinis | ATCC 55755 | prokaryotic |
| Synechococcus sp. | PCC 7942 | prokaryotic |
| Synechococcus elongatus | UTEX LB 563 | prokaryotic |
| Synechococcus leopaliensis | UTEXB 2434 | prokaryotic |
| Synechococcus sp. | ATCC 27147 | prokaryotic |
| Synechococcus sp. | PCC 7003 | prokaryotic |
| Synechococcus sp. | ATCC 27179 | prokaryotic |
| Fremyella diplosiphon | UTEX 481 | prokaryotic |
| Fremyella diplosiphon | UTEX B 590 | prokaryotic |
| Synechocystis nigrescens | UTEX LB 2587 | prokaryotic |
| Synechocystis sp. | UTEX B 2470 | prokaryotic |
| Synechocystis sp. | PCC 6804 | prokaryotic |
| | ATCC 27185 | |
| Synechocystis sp. | ATCC 29110 | prokaryotic |
| Synechocystis sp. | PCC 6803 | prokaryotic |
| | ATCC 27184 | |

^{†}UTEX refers to strains from the algae collection of the University of Texas (Austin, TX); CC-refers to strains from the algae collection of the Chlamydomonas Genetics Center at Duke University (Durham, NC); ATCC refers to strains from the algae collection of the American Type Culture Collection (Manassas, VA).

Wild type and non-wild type starting strains can be used as host organisms for expression of light utilization alteration constructs. Non-wild type starting strains can exhibit a specific desirable phenotype regardless of whether or not the identity or location of one or more genes that have been altered to cause the phenotype are known.

An example of a construct that alters the phenotype of cells is an iron hydrogenase expression construct containing an amino acid substitution that confers oxygen-tolerant hydrogen production (see U.S. patent application Ser. No. 10/763,712). Another example is a construct that encodes an eyme that participates in the biosynthetic pathway of a terpenoid molecule such as taxol (see Proc Natl Acad Sci U S A. 2004 Jun. 15;101(24):9149-54).

Another example of a non-wild type strains is a strain that is deficient in one or more aspects of motility. Such mutants contain genetic alterations in one or more genes that regulate flagella structure and/or function. The genetic alterations that cause deficiencies in motility can be known or unknown. Many C. reinhardtii strains are known to be partially or completely deficient in motility, such as pf6 (CC-929, CC-1029), pf16 (CC-624, CC-1024), pf20 (CC-22, CC-261), pf24, (CC-1384, CC-2500), pf4 (CC-613), pf17 (CC-262), pf26 (CC-1386), pf1 (CC-602), pf3 (CC-604), pf4 (CC-680) and other paralyzed strains. Other strains that have reduced or eliminated motility are described as BOP1, BOP2, BOP3, BOP4, BOP5, CPC1, ENH1, FLA1, FLA2, FLA3, FLA4, FLA5, FLA6, FLA8, FLA9, FLA10, FA11, FLA12, FLA13, IDA2, IDA3, IDA4, LF1, LF2, LF3, LIS1, LIS2, MBO1, MBO2, MBO3, ODA1, ODA2, ODA3, ODA4, ODA5, ODA6, ODA7, ODA8, ODA9, ODA10, ODA11, PF2, PF4, PF5, PF7, PF8, PF9, PF10, PF12, PF13, PF15, PF18, PF19, PF21; PF22, PF23, PF25, PF27, PF29, SHF1, SHF2, SHF3, SPF2, SPF3, SUN1, TNR1, UNI1, VFL1, VFL2 and VFL3. A high level of detail about these mutants, including strain numbers, can be found under the “Motility Impaired” phenotypic classification in the chlamyDB database of the Chlamydomonas Genetics Center, Duke University (http://www.biology.duke.edu/cgi-bin/ace/searches/browser/default).

Motility mutants can also be made conditionally paralyzed by the inducible expression of RNAi or antisense constructs that target transcripts of flagella genes. Some of the genes mutated to cause the above described motility impairment phenotypes in C. reinhardtii have been characterized (see for example Eukaryot Cell. 2004 Aug. 3(4):870-9; Cell Motil Cytoskeleton. 2000 July; 46(3):157-65; Mol Biol Cell. 1997 Mar. 8(3):455-67; J Cell Biol. 1986 July; 103(1): 1-11)). The sequences of these genes can be used to construct RNAi or antisense expression vectors through operable linkage with promoters.

Chlorella species have no flagella and are therefore naturally incapable of exhibiting flagella-based motility. Strains of Volvox with impaired motility are known (J Cell Sci. 2000 December; 113 Pt 24:4605-17).

Paralyzed cyanobacterial strains are also known (for examples, see Plant Cell Physiol. 2001 January; 42(1):63-73 and Mol Microbiol. 2000 August; 37(4):941-51 (Synechocystis PCC 6803); Proc Natl Acad Sci U S A. 1996 Jun. 25;93(13):6504-9 (Synechococcus sp. strain WH8102); Plant Cell Physiol. 2002 May; 43(5):513-21 and Photochem Photobiol Sci. 2004 Jun. 3(6):503-11 (Anabaena).

A plurality of starting strains can also be used in the methods provided herein. For example, two or more starting strains can be simultaneously transformed with a light utilization alteration construct or a library of light utilization alteration constructs before the screening or selection step. For example, motility deficient C. reinhardtii mutant strains CC-929, CC-624, CC-261, CC-1384, CC-613, CC-262, CC-1386, CC-602, CC-604, and CC-680 can be cultured to a stable cell concentration and measured. From the cell concentration measurements using a hemocytometer or optical density measurements, an equal number of cells of each strain are mixed into a tube shortly before the transformation reaction with a library of light utilization alteration constructs.

### B. Transformation Methods

In Chlamydomonas, the nuclear, mitochondrial, and chloroplast genomes are transformed through a variety of known methods. (Kindle, J Cell Biol (1989) December; 109(6 Pt 1):2589-601; Kindle, Proc Natl Acad Sci U S A (1990) February; 87(3):1228-32; Kindle, Proc Natl Acad Sci U S A (1991) Mar. 1;88(5):1721-5; Shimogawara, Genetics (1998) April; 148(4):1821-8; Boynton, Science (1988) Jun. 10;240(4858):1534-8; Boynton, Methods Enzymol (1996) 264:279-96; Randolph-Anderson, Mol Gen Genet (1993) January; 236(2-3):235-44).

Transformation methods for other eukaryotic microalgae are also known (see for example Curr Microbiol. 1997 December; 35(6):356-62 (Chlorella vulgaris); Mar Biotechnol (NY). 2002 Jan. 4(1):63-73 (Chlorella ellipsoidea); Mol Gen Genet. 1996 Oct. 16;252(5):572-9 (Phaeodactylum tricornutum); Plant Mol Biol. 1996 April; 31(1):1-12 (Volvox carteri); Proc Natl Acad Sci U S A. 1994 Nov. 22;91(24): 11562-6 (Volvox carteri); Falciatore A, Casotti R, Leblanc C, Abrescia C, Bowler C, PMID: 10383998, 1999 May 1(3):239-251 (Laboratory of Molecular Plant Biology, Stazione Zoologica, Villa Comunale, I-80121 Naples, Italy) (Phaeodactylum tricornutum and Thalassiosira weissflogii)).

Transformation methods and selectable markers for cyanobacteria are known in the art (Koksharova, Appl Microbiol Biotechnol 2002 February; 58(2):123-37 (various species); Mol Genet Genornics. 2004 February; 271(1):50-9 (Thermosynechococcus elongates); J. Bacteriol. (2000), 182, 211-215; FEMS Microbiol Lett. 2003 Apr. 25;221(2):155-9; lant Physiol. 1994 June; 105(2):63541; Plant Mol Biol. 1995 Dec. 29(5):897-907 (Synechococcus PCC 7942); Mar Pollut Bull. 2002;45(1-12):163-7 (Anabaena PCC 7120); Proc Natl Acad Sci U S A. 1984 March; 81(5):1561-5 (Anabaena (various strains)); Proc Natl Acad Sci U S A. 2001 Mar. 27;98(7):4243-8 (Synechocystis); Wirth, Mol Gen Genet 1989 March; 216(1):175-7 (various species); Mol Microbiol, 2002 June; 44(6):1517-31 and Plasmid, 1993 Sep. 30(2):90-105 (Fremyella diplosiphon). Anabaena species are sometimes referred to in the scientific literature as Nostoc.

### C. Placing Transformants into Culture Containers

After transformation with one or more light utilization alteration constructs, colonies that contain a selectable or screenrable marker, and therefore the construct, are identified and can be placed into a culture container for screening or selection for a desired function. It is preferred but not required that the cells be screened or selected for a desired function while in liquid culture media. If a library of light utilization alteration constructs is used to transform the organism, a plurality of colonies containing different members of the library are preferably arrayed into multiwell plates.

Preferably, a culture container used for screening and selection, including a multiwell plate, is made of substantially nontransparent material. Nontransparent material means materials that allows no more than 80% of photons to pass through, more preferably no more than 40%, more preferably no more than 20%, more preferably no more than 10%, more preferably no more than 5%, more preferably no more than 2%, and more preferably no more than 0.01% at a light intensity of 25-1000 μmol photons m⁻² s⁻¹. Most preferably, the culture container allows no light to pass through at a light intensity of 1000 μmol photons m⁻² s⁻¹. Independent transformant strains initially plated on solid growth media can be arrayed into multiwell plates manually or using a robot. Cells arrayed into culture containers, preferably made of nontransparent materials, are then assayed in a format where they receive light only from above the plane of the culture media surface. The use of nontransparent materials ensures that the cells receive light only from above. This assay format mimics the conditions of an outdoor bioreactor where cells receive light only from a single overhead light source (the sun). Multiwell plates made of substantially nontransparent material are commercially available (see for example VWR catalog number 29444-018 (manufactured by Costar); and Fisher Scientific catalog number 14-245-176 (manufactured by Thermo Electron Corporation, Milford, Mass.),

It is preferred that the cells in a culture container be present in liquid culture media In addition, it is preferred that enough cells are present in the culture container that a plurality of layers of cells is present, as shown in FIGS. 5, 6 and 11. When colonies are initially identified from solid growth media, it is preferred that enough cells be transferred to the culture container that a plurality of layers of cells are created in the culture container such as a well of a multiwell plate. Alternatively, cells transferred from solid growth media to the culture container can be cultured for a period of time ranging from at least 30 minutes to several months or longer to allow the cells to divide to generate a plurality of layers of cells. The number of cells it takes to form a plurality of layers of cells is a function of cell size, maximum cell density, and the total area of the surface of the culture media. It is of course not necessary that the cells form discrete layers of cells, but rather it is preferred that there are enough cells in a culture container that there are cells that are not at the surface of the culture media. If the cells not capable of motility and are on the bottom of a culture container it is preferred that there be enough cells to completely cover the cells touching the bottom surface of the culture container.

### IV Screening

### A. General Screening Methods

Cells transformed with light utilization alteration constructs can be screened for the ability to perform one or more functions that require energy.

i. Photosynthesis Indicators

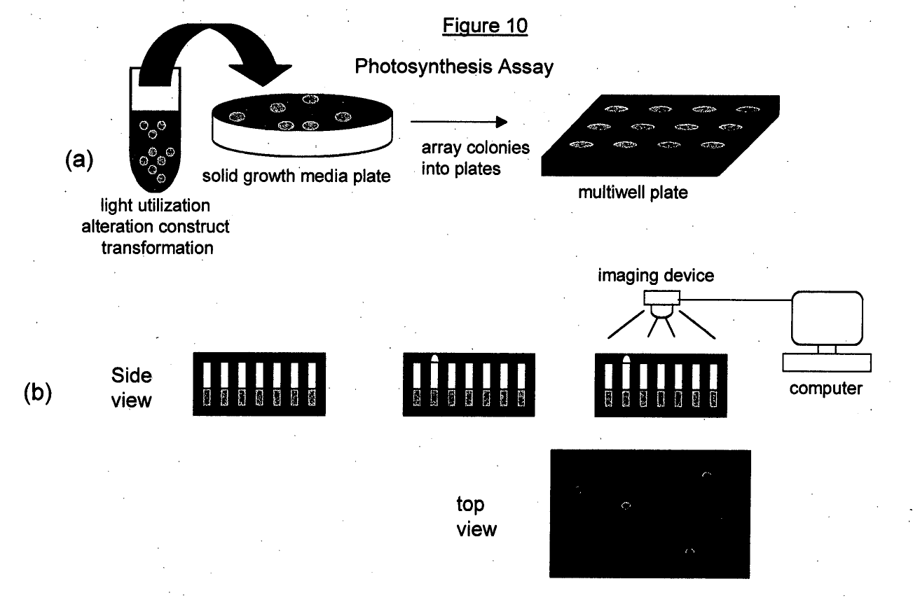

Cells can be screened for the ability to produce molecules in photosynthesis-driven reactions. For example, cells can be assayed for the ability to generate maximal amounts of oxygen when exposed to light. Methods for detecfion of oxygen are known. For example, oxygen production can be measured through gas chromatography, and other methods (see oxygen analyzers from Advanced Micro Instruments Inc., for example). Alternatively, chemochroric films containing transition metals and a palladium catalyst layer can be used to assay for oxygen production. This is performed by placing a chemochromic film (as described in U.S. Pat. Nos. 6,277,589 and 6,448,068) in saturating concentrations of hydrogen gas to turn the film from transparent to dark. The saturated film is then placed, for example, on top of a multiwell plate containing cells transformed with a library of light utilization alteration constructs that have been exposed to light before the film is placed on top of the multiwell plate as depicted in FIG. 10. Oxygen produced by photosynthetic water splitting diffuses into the gas space above the cells and contacts the film. Oxygen competes for binding with hydrogen to the film, displacing bound hydrogen atoms and “bleaching” the film. Cells in wells that are more proficient at utilization of absorbed light produce more oxygen and produce the lightest spots on the film.

Another assay that can be performed to measure photosynthetic output is ATP production. It is preferred that ATP production is measured by cells that are not exposed to any energy source other than light. ATP assays are known and are commercially available (see Mol Gen Genet. 1999 October; 262(3):421-5; ATP Kit SL Prod No. 144-041, BioThema Inc., Handen, Sweeden; Steady-Glo® Luciferase Assay System, Promega Inc., Palo Alto, Calif.; LBR-T100, proteinkinase de, Kassel, Germany; BO1243-107, Thermo Electron Corporation, Milford, Mass.).

Cells can be assayed for ATP production by culturing the cells and measuring ATP concentration. An example of an assay system is expression of an ATP-consuming protein in the cell, where ATP consumption can be measured through biolumninescence. As an example, luciferase proteins consume ATP as an energy source for generating detectable light. A luciferase gene can be cloned into a cell, preferably using the preferred codons of the host in the nucleotide sequence of the luciferase. In a preferred embodiment, the luciferase gene is inducible and is present in the starting strain used to generate a library of organisms, each independent transformant containing at least one light utilization alteration construct library. After the cells are cultured under light after being placed in a multiwell plate made of nontransparent material, expression of the luciferase gene is induced. The cells are then assayed in the dark for light emission. Strains in wells of the plate that generate more light have more ATP available and utilize light more efficiently as a population. Luciferase genes are known, as well as inducible systems such as the tetracycline repressor-activator system (Pigment Cell Res. 2004 Aug. 17(4):363-70; PLoS Biol. 2004 Jun. 2(6):763-75; Methods Mol Biol. 2004;270:287-98).

A luciferase gene can be cloned into the chloroplast genome of a eukaryotic photosynthetic microorganism in a specifically desired location Firefly luciferase, for example, catalyzes the oxidation of luciferin in the presence of ATP, magnesium ions and molecular oxygen with a high quantum yield. Due to its high sensitivity and specificity for ATP, luciferase has been used for bioluminescent detection of ATP in various biological samples. Preferably the luciferase gene is targeted to a position in the chloroplast genome that does not interfere with the expression of other genes. The promoter driving the luciferase gene is preferably inducible but based on a known chloroplast promoter sequence such as atpA or psbA (see Plant J. 2004 February; 37(3):449-58 and J Biolumin Chemilumin. 1989 Jul. 4(1):375-80 for Chiamydomonas chloroplast expression and a review of luciferase technology, respectively).

An alternative to expression of a luciferase gene in an ATP assay is to add luciferase protein directly to cells before lysis or to lysates. In this method, cells are typically cultured in multiwell plates for a certain period of time and then subjected to centrifugation, followed by removal of culture media. The cells are then lysed using chemical, mechanical, or other means, followed by addition of luciferase protein, buffers, and other reagents. The amount of ATP in each well containing lysed cells is then measured, for example, using a luminometer. ATP can also be extracted from cells using trichloroacetic acid, followed by neutralization of pH and addition of luciferase protein. Other energy containing molecules such as GTP can also be assayed.

Cells can also be screened for reduced chlorophyll fluorescence. Assays for reduced chlorophyll fluorescence are known (Planta 2003 May; 217(1):49-59) and can be used with any photosynthetic microorganism.

ii. Other Molecules Produced Using Photosynthetic Energy

The production of a molecule requires chemical energy, and as a result, production of a particular molecule can be measured as a means to detect increased light utilization efficiency.

Carotenoids are naturally synthesized by photosynthetic microorganisms, and are a subset of a class of molecules known as isoprenoids. Production of carotenoids can be measured as a means to detect increased light utilization efficiency. Carotenoids that can be measured include zeaxanthin, astaxanthin, annatto (bixin/norbixin), β-carotene, β-apo-8-carotenal, β-apo-8-carotenal-ester, and capsanthin. Carotenoids can be measured using techniques such as HPLC (Biol Res. 2003;36(3-4):343-57; Biol Res. 2003,36(2):185-92), Raman spectroscopy (Appl Spectrosc. 2004 April; 58(4):395-403; J Biomed Opt 2004 Mar.-Apr. 9(2):332-8; J Biomed Opt 2002 Jul. 7(3):435-41), and mass spectroscopy (J Chromatogr A. 1999 Aug. 27;854(1-2):233-44; Methods Enzymol. 1997;282:130-40).

Some wild type photosynthetic microorganisms can produce hydrogen gas, such as Chlamydomonas reinhardtii, Chlamydomonas moewusii, Scenedesmus obliquuus, and others. Other photosynthetic microorganisms can be engineered to produce hydrogen. When these photosynthetic microorganisms are cultured on minimal growth media containing no energy source, light is the only energy containing nutrient available. Populations of microorganisms genetically programmed to generate hydrogen can be exposed to bright light conditions and assayed for hydrogen production. Enhanced light utilization caused by light utilization alteration constructs is detected through increased hydrogen production.

Hydrogen may be detected using a variety of methods such chemochromic sensing films that contain transition metals (see U.S. Pat. No. 6,277,589). Such films change from clear to dark grey-blue when exposed to hydrogen, and when placed in proximity to cells that produce different amounts of hydrogen they identify cells that produce more hydrogen than others. There are other methods, both direct and indirect, that are used to detect hydrogen, such as spectroscopic methods (see U.S. Pat. Nos. 5,100,781 and 6,309,604). Other types of gas sensors and films suitable for detection of hydrogen are known in the art (see U.S. Pat. Nos. 5,100,781, 6,484,563, 6,265,222 and 6,006,582).

For example, a transition metal-containing chemochromic film is placed on top of a multiwell plate made of nontransparent material containing liquid culture media, with one or more wells containing one or more independent transformants containing at least one light utilization alteration construct. The film is placed against the plate such that each well is sealed or partially sealed from the outside atmosphere. Preferably the culture media does not fill the well so that a space of gas separates the media from the film. The amount of color change in the film at each spot above a culture well is then measured, preferably in a quantitative fashion, using techniques such as densitometry or other scanning methods. Alternatively, a digital camera photographs the film immediately after exposure to the transformed cells. Films may also be analyzed by visual inspection. Parameters such as the length and intensity of light exposure before the film is placed over the culture wells for the hydrogen assay may be varied. For example, strains that are capable of sustained hydrogen production over the course of a 12 hour period in which the intensity of light is increased and decreased to roughly correspond to daylight may be isolated by performing the hydrogen assay after the cells have been producing hydrogen for a desired number of hours.

Production of a recombinant protein can be measured as a means to detect increased light utilization efficiency. Assays for production of a recombinant protein are known, and typically use an antibody that specifically recognizes the recombinant protein.

For example, production of human insulin by photosynthetic microorganisms transformed with one or more light utilization alteration constructs can be detected. Antibodies to human insulin are commercially available (Linco Research Inc., St. Charles, Mo., Catalog #: 1014; Research Diagnostics Inc., Flanders, N.J.; Serotec, Oxford, U.K, catalog no. MCA1911G). Antibodies are typically immobilized on a solid substrate such as the wells of a multiwell plate. Cells producing insulin are lysed, and insulin from the cells is bound by antibodies immobilized to the plate and detected. Immunoassay technology is known in the art (see for example, U.S. Pat. Nos. 6,143,511, 6,048,705, 5,973,123 and 5,925,533).

In a preferred embodiment, a plurality of strains that exhibit increased light utilization efficiency are identified. Cells from each strain are placed together and induced to mate. The progeny are screened for the ability to utilize light more efficiently than any parental strain. Strains may be mated in a pairwise (2 strains) or multiparental (3 or more strains) fashion. Methods for mating photosynthetic microorganisms are known (see for example (Harris, (1989) The Chlamydomonas Sourcebook. Academic Press, New York).

It should be apparent to one skilled in the art that various embodiments and modifications may be made to the invention disclosed in this application without departing from the scope and spirit of the invention. All publications mentioned herein are cited for the purpose of describing and disclosing reagents, methodologies and concepts that may be used in connection with the present invention Nothing herein is to be construed as an admission that these references are prior art in relation to the inventions described herein. All publications cited are incorporated by reference in their entirety for all purposes.

### EXAMPLE 1

Starting Strain: Chlamydomonas reinhardfii strain CC-124 (Chlamydomonas Genetics Center, Duke University) is cultured and maintained in TAP media (Harris, 1988) unless otherwise specified.

Luciferase Transformation of Chloroplast: The chloroplast genome of the starting strain is transformed with a bacterial luciferase expression vector, as described in Mayfield, Plant J. 2004 February; 37(3):449-58. A gene encoding the bacterial luciferase protein luxCt (Genbank accession number AY366360), encoded by the C. reinhardtii chloroplast most preferred codons (see http://www.kazusa.or.jp/codon/), is placed in operable linkage with the AtpA promoter and the 3′ UTR of the rbcL gene. As described in Mayfield, the construct is cloned into the chloroplast transformation vector p322, which contains a spectinomycin resistance gene (see Methods Mol Biol. 2004;274:301-8). Spectinomycin resistant clones are tested for functional luciferase expression by using a CCD camera, as described in Mayfield. The luciferase expressing strain is referred to herein as 124-luc.

Light Utilization Alteration Construct Promoters: The promoter section of the light utilization alteration construct is constructed as a library of promoter sections amplified by PCR from the genomic C. reinhardtii sequence upstream of the coding regions of the genes listed in table 1. The promoter sequences are amplified as shown schematically in FIG. 8, creating promoter sequences of 9 different lengths. Because the amount of sequence between the start of transcription and the start of translation varies in each gene, the length of the 9 fragments generated for each promoter varies, however the three sense primers are designed to anneal upstream of the TATA box and the three antisense primers are designed to anneal downstream of the start site of transcription. The amplification strategy is depicted in FIG. 9A for the light activated Mg chelatase ChlI subunit gene promoter. Sense primer sequences are underlined while the antisense primers are underlined and italicized. The same scheme in FIG. 9B depicts the amplification and primer design for the light activated phosphoglycerate kinase gene promoter.

Both antisense and sense primers used for amplification of promoter fragments have 5′ linker tail sequences that do not correspond to the promoter sequence the 3′ region anneals to. Linker tail sequences allow the amplified fragments to be connected to other segments of the light utilization alteration construct. All sense promoter primers use the same 5′ tail sequence (SEQ ID NO: 17). All antisense promoter primers have the same 5′ tail sequence (SEQ ID NO: 18). The tail sequence of the antisense promoter primer is complementary to the upstream end of the light utilization alteration segments described below. The tail sequence of the sense primer is complementary to the upstream end of the promoter that drives the selectable marker gene, also described below.

The amplification reactions are performed as follows: Primers for amplifying 9 lengths of primer sequence from the promoters of the genes listed in table 1 are synthesized chemically and obtained from commercial sources (BioNeuus Inc., Oakland, Calif.). The primers, exemplified by SEQ ID NOs: 11-13 (sense for the Mg chelatase ChlI subunit gene promoter), SEQ ID NOs: 14-16 (antisense for the Mg chelatase ChlI subunit gene promoter) are placed into PCR reactions containing standard components (0.2 mM of each dNTP, 2.2 mM MgCl₂, 50 mM KCl, 10 mM Tris-HCl pH 9.0, 0.1% Triton X-100, 2.5 units of Pfu polymerase). Approximately 100 ng of C. reinhardtii genomic DNA is added to the reaction as template. Isolation protocols for generating C. reinhardtii genomic DNA are known (Harris, 1989). The themocycling program contains a single denaturation at 94° C. for 60 seconds, followed by 40 cycles of 94° C. for 30 seconds, 62° C. for 30 seconds, and 72° C. for 30 seconds, followed by a one time incubation of 72° C. for 5 minutes.

The amplification scheme depicted in FIG. 8 yields the PCR products described in Table 3. Amplification of nine promoter fragments from each of the 47 promoters of the genes listed in table 1 yields a total of 423 promoter fragments.

The PCR products from all reactions are purified via agarose gel electrophoresis and electroelution from gel fragments. The electroeluted PCR products are precipitated from the electroelution buffer with 0.5 volumes of 7.5 M NH₄OAc and 2 volumes of −20° C. 100% ethanol. The products are then are pelleted at 14,000×g. The pellets are washed two times with −20° C. 70% ethanol. The pellets are dried and resuspended in water.

### Light Utilization Alteration Segments:

The light utilization alteration segments contain a linker tail segment complementary to the antisense linker tail segment of the primer used to amplify promoter segments (Linker 2, FIG. 1, SEQ ID NO: 19), a sense segment identical to a segment of a gene encoding a protein involved in light harvesting from Table 7, a loop segment (SEQ ID NO:23), an antisense segment complementary to the sense segment, and a transcription termination segment (SEQ ID NO:58). These segments are positioned in the order listed above and shown in FIGS. 1-3.

The sense sequences of the RNAi segments are shown below in Table 7. Because light harvesting polypeptide genes such as those listed in table 2 have a high level of nucleotide sequence similarity with each other, it is possible to design RNAi segments that target a plurality of members of a gene family (multitargeting segments). Some multitargeting sense segments target all members of a gene family, such as the segment containing SEQ ID NO:31. Because light harvesting polypeptide genes also contain sequence variability, it is also possible to design RNAi segments that target only one member of a family (single targeting segments).

TABLE 5
Sense sequences of multitargeting or single targeting RNAi molecules

| Genes | | |
|---|---|---|
| containing | Multitargeting or Single | |
| segment | targeting sense segment | Nucleotide position of segment in full length cDNA |
| LI818-1 and | gcagatcggccagggcttctggga | 369-392 of LI818-1 coding region; 198-221 of LI818-2 |
| LI818-2 | SEQ ID NO:33 | coding region |
| Lhca2 and | aaggaggtcaagaacggccgcctgg | 565-589 of Lhca2 coding region; 469-493 of Lhca7 |
| Lhca7 | SEQ ID NO:22 | coding region |
| Lhca4 and | tcaagaacggccgcctggccatggt | 533-557 of Lhca4 coding region; 476-500 of Lhca7 |
| Lhca7 | SEQ ID NO:34 | coding region |
| Lhcbm3 and | ggccccaaccgcgccaagtggctgg | 124-148 of Lhcbm3 coding region; 115-139 of Lhcbm9 |
| Lhcbm9 | SEQ ID NO:35 | coding region |
| Lhcbm1 and | gactacggctgggacaccgccggtc | 202-226 of Lhcbm1 coding region; 202-226 of Lhcbm3 |
| Lhcbm3 | SEQ ID NO:36 | coding region |
| Lhcbm6 | ggctgggcccctactctgagaacg | 131-154 of coding region |
| | SEQ ID NO:25 | |
| LI818-1 | gagctgaagaccctgcagacc | 547-567 of coding region |
| | SEQ ID NO:37 | |
| LI818-2 | gagctcaaggtcatgcagacc | 376-396 of coding region |
| | SEQ ID NO:38 | |
| tIa1 | tcgcccaggtggagtcctacac | 191-212 of coding region |
| | SEQ ID NO:27 | |
| Mg chelatase | gtggtgtcatgatcatgggcg | 311-331 of coding region |
| subunit I | SEQ ID NO:29 | |
| Lhcbm1-7 and | tacctgactggcgagttccccgg | |
| Lhcbm 8-9 | SEQ ID NO:31 | |

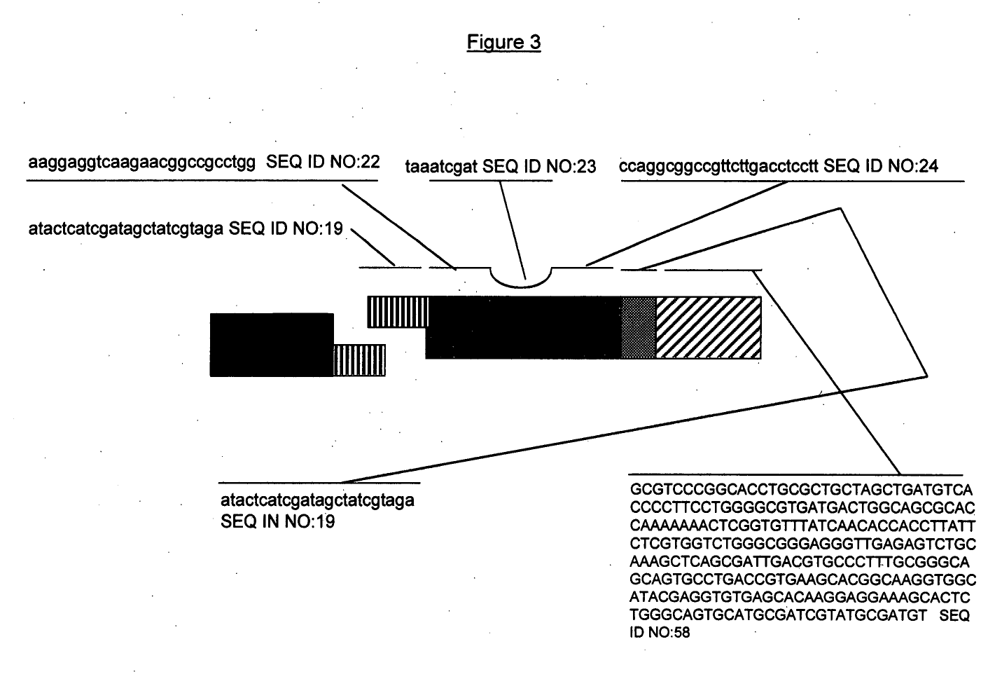

Light utilization alteration segments are generated by chemical synthesis. For example, the light utilization alteration segment targeting the Lhca2 and Lhca7 genes is shown in FIG. 3 (SEQ ID NO:21), and this segment in operable linkage with a transcription termination segment is SEQ ID NO:59. A light utilization alteration segment is generated for each of the sense strands from table 5 and their antisense counterparts with a loop section (SEQ ID NO:23) separating them, as shown in FIGS. 1 and 3.

The light utilization alteration segments are synthesized as double stranded DNA molecules by primeness PCR of 20-40 mer oligonucleotides encoding both strands of the entire light utilization alteration segment and a transcriptional termination sequence as described in Gene, 1995 Oct. 16;164(1):49-53. The exemplary light utilization alteration segment and a transcriptional termination sequence targeting the Lhca2 and Lhca7 gene is SEQ ID NO:59. Primerless PCR products are purified via agarose gel electrophoresis and electroelution from gel fragments. The electroeluted segments are precipitated from the electroelution buffer with 0.5 volumes of 7.5 M NH₄OAc and 2 volumes of −20° C. 100% ethanol. The products are then are pelleted at 14,000×g. The pellets are washed two times with −20° C. 70% ethanol. The pellets are dried and resuspended in water.

Selectable Marker Gene: A ble selectable marker gene cassette (SEQ ID NO:55), including an RBCS2 promoter and RBCS2 3′ untranslated region (SEQ ID NO:60) operably linked to the ble cDNA, includes a linker tail cormplementary to Linker 1. The promoter-ble cassette contains the linker at its upstream end, as shown in FIG. 1. (also see Mol Gen Genet. 1996 Apr. 24;251(1):23-30 and Plant J. 1998, 14, 441-448 for details of the ble marker).

The ble selectable marker gene cassette is generated via primerless PCR from 20-40 mer oligonucleotides encoding both strands of SEQ ID NO:55 and the PCR product is purified via agarose gel electrophoresis and electroelution from gel fragments. The PCR product is precipitated from the electroelution buffer with 0.5 volumes of 7.5 M NH₄OAc and 2 volumes of −20° C. 100% ethanol. The product is then are pelleted at 14,000×g. The pellets are washed two times with −20° C. 70% ethanol. The pellets are dried and resuspended in water.

Synthesis of Library of Light Utilization Alteration Constructs: The light activated promoter segments, light utilization alteration segments, and selectable marker are used to construct a library of light utilization alteration constructs as follows:

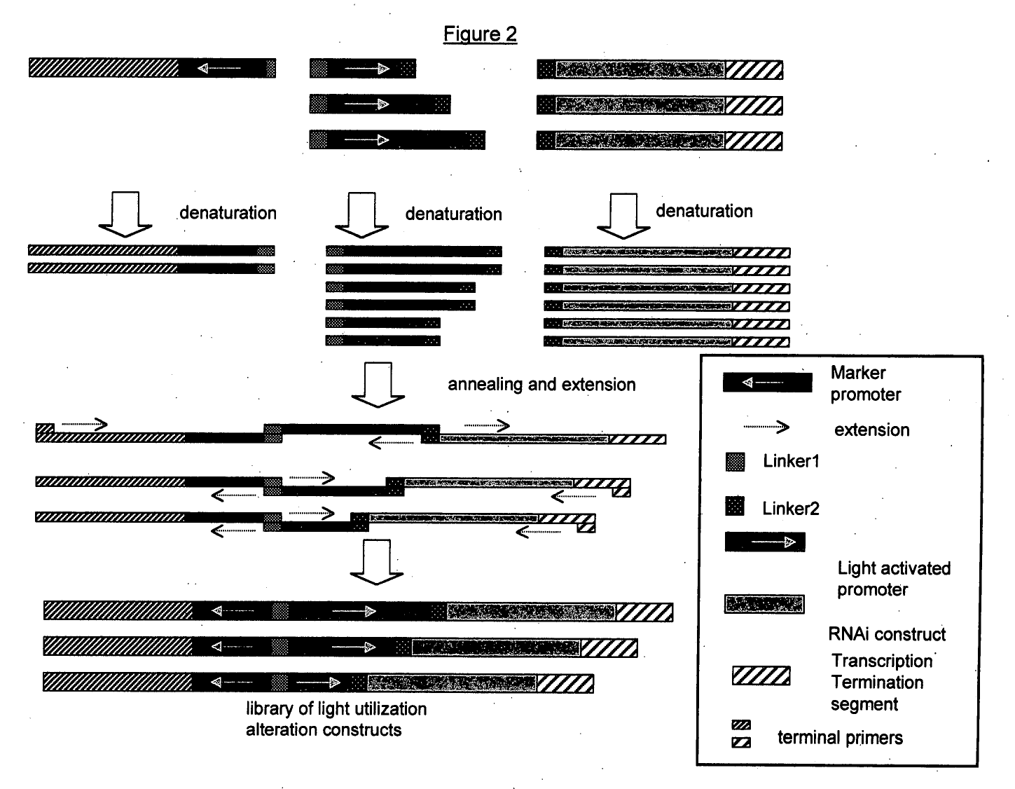

100 μmol of single stranded terminal primers, double stranded light activated promoter segments, double stranded light utilization alteration segments including transcriptional termination segments, and double stranded ble selectable marker cassettes are placed into a single reaction and subjected to PCR (as shown in FIG. 2). The tube is heated at 95° C. for 5 minutes. The reaction is then cooled to 65° C. for 30 seconds and then heated to 72° C. for 2 minutes. 30 cycles of 1 minute at 95° C., 30 seconds at 65° C., and 2 minutes at 72° C. are then performed. The PCR products are gel purified, electroeluted, phenol:chloroform extracted, precipitated and resupended. The due to variability of the size of the light activated promoter fragments, the light utilization alteration construct library comprises individual constructs of varying sizes. A representative member of the library is SEQ ID NO:56, with the exception that the ble marker gene and promoter in SEQ ID NO:56 is in hfie opposite orientation as shown in FIGS. 1 and 2. This construct contains (1) the ble gene, conferring resistance to phleomycin, in operable linkage with the promoter and transcriptional termination region of the RBCS2 gene; and (2) a fragment of the Mg chelatase promoter in operable linkage with the light utilization alteration segment (the RNAi segment targeting the Lhca2 and Lhca7 genes), which is in turn in operable linkage with the transcriptional termination region of the histone H3 gene.

Transformation of 124-luc strain to generate light utilization alteration library: The 124-luc strain is transformed with the library using the glass bead method of transformation (Kindle 1990 Proc. Natl. Acad. Sci. USA 87, 1228-1232) to yield a library of independent colonies referred to herein as 124 luc-lual Clight utilization alteration library). The transformation reaction is plated on solid TAP media (Harris E H (1989) The Chlamydomonas Source Book. Academic Press, San Diego). Individual colonies are picked and arrayed by optical robot (Genetix USA Inc., Boston, Mass.). The colonies are arrayed into 96 well deep well plates made of dark, nontnansparent plastic (Thermo Electron Corporation, Milford, Mass.). The liquid media in the multiwell plates is Sager's minimal media (Harris, 1989), each well containing 400 ul of media 10,000 colonies are picked and arrayed into 108 plates, including 3 control wells on each plate containing the 124-luc strain.

ATP Assay: The multiwell plates containing the light utilization alteration library of independent 124-luc-lual strains are placed under constant light (800 μmol s⁻¹ m⁻¹) for 5 days and held under constant temperature at 30° C. After 5 days, decanal (0.1%, Signa Aldrich, St. Louis, Mo.) is swabbed onto the underside of the lid of each plate. After decanal addition, each plate is placed in the dark for 5 minutes to eliminate chlorophyll fluorescence. Each plate is then assayed for ATP concentration using a charged coupled device (CCD) camera, as described in Mayfield, Plant J. 2004 February; 37(3):449-58.

Strains that generate a higher luciferase signal than the 124-luc strain are selected for further development. Optionally, multiple strains that exhibit a luciferase signal are subjected to pairwise or multiparental mating protocols followed by an additional ATP assay to identify further improved strains. Mating protocols are disclosed, for example, in U.S. patent application Ser. No. 10/763,712 and Harris, 1989.

### EXAMPLE 2

Starting Strain: Synechococcus sp. strain WH8102 (Proc Natl Acad Sci U S A. 1996 Jun. 25;93(13):6504-9) is cultured in BG11 medium (Methods Enzymol. (1988) 167, 100-105). Cultures (50 ml) in 125-ml flasks are incubated without shaking at 25° C. and with constant illumination (10 μE/m⁻²/sec⁻¹) unless otherwise indicated.

Light Utilization Alteration Constructs: Constructs are generated by primeriess PCR of 40-mer oligonucleotides encoding the constructs of SEQ ID NOs: 50-52 (Gene. 1995 Oct. 16;164(1):49-53).

The promoter placed in operable linkage with the light utilization alteration segment is the Synechococcus htpG gene light activated promoter (SEQ ID NO:39). The light utilization alteration segments used in constructs of SEQ ID NOs: 50, 51, and 52 target the Synechococcus allophycocyanin beta-18 subunit, CP43 and chlorophyll synthase genes, respectively. The transcription terminator segrnent in operable linkages with the antisense constructs is a tandem repeat of the terminator sequence of Synechococcus 7942 gap2 gene. The promoter in operable linkage with the spectinomycin resistance gene is a section of the Synechococcus ribulose-1,5-bisphosphate carboxylaseloxygenase promoter. The streptomycin resistance cDNA (streptomycin adenylyltransferase cDNA) corresponds to GenBank accession number AF424805. The transcription terminator in operable linkage with the streptomycin adenylyltransferase gene is the termiator sequence of Synechococcus ribulose-1,5-diphosphate carboxylase gene (Genbank accession number E14860).

TABLE 6
Light Utilization Alteration Segments:

| | Accession | | |
|---|---|---|---|
| | number of | | |
| | full length | | |
| Target Gene | gene | Antisense sequence | Function |
| Allophycocyanin | BX569692 | gaggaaatagtccatatcccgcag | Antenna |
| beta-18 subunit | | acaggccgcgaggcgtctggtggt | |
| | | gtaggcattcccaccagggagga | |
| | | gaagttccggctcaatcac | |
| | | SEQ ID NO:41 | |
| CP43 | NC_005070 | ggtacagaccgcccaggccgag | Antenna |
| | | aacggcagagctgatcaggtgca | |
| | | gaacaccgaccacgaa | |
| | | SEQ ID NO:43 | |
| chlorophyll | NC_005070 | ccagtccggcgaggctgtaggca | Chlorophyll |
| synthase | | agggtcagcagagccgtgctcca | biosynthesis |
| | | ggtcagttggccgaa | |
| | | SEQ ID NO:45 | |

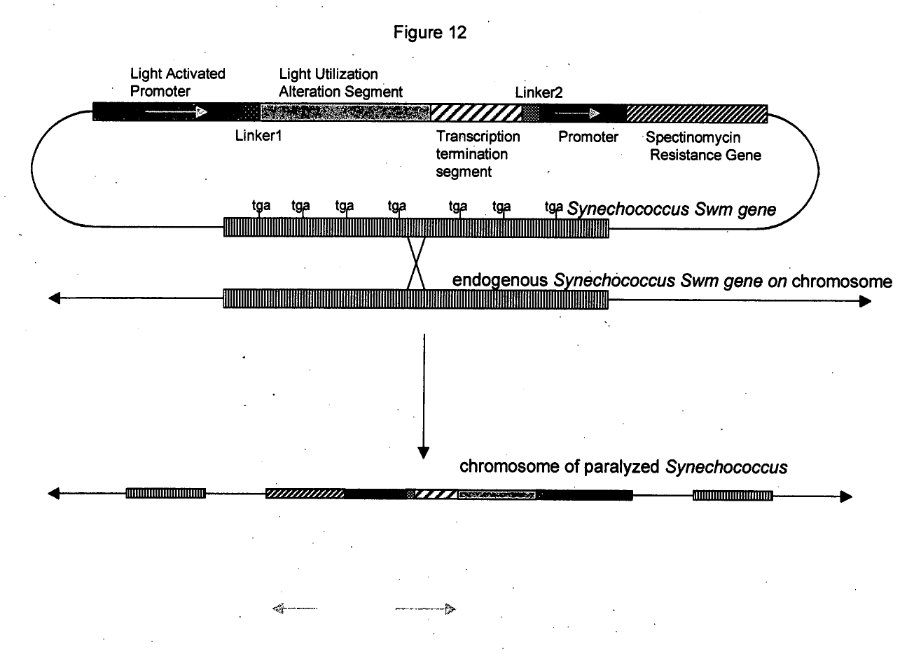

Homologous Recombination Section: A nucleotide sequence encoding the Synechococcus Swm gene, including seven in-frame stop codons, is generated by primeness PCR of 40-mer oligonucleotides encoding SEQ ID NO:53. The section is cloned into a separate circular plasmid containing the light utilization alteration constructs of SEQ ID NOs:50-52, as depicted in FIG. 12. Numerous plasmids are available for transformation of Synechococcus, cited above.

Transformation: Synechococcus sp. strain WH8102 cells are transformed according to the method of Methods Enzymol. 1987; 153:215-31 and are plated on solid BG-11 medium.

Streptomycin resistant colonies containing each light utilization alteration construct described above are picked from solid media plates and cells from ten independent colonies containing each light utilization alteration construct are placed into deep well plates made of dark, nontransparent plastic (Thermo Electron Corporation, Milford, Mass.) containing liquid ASN II medium (Arch Mikrobiol., 1972 87:93-98.), modified to include 15 mM TES (N-tris(hydroxymethyl)methyl-2-amino ethanesulfonic Acid) as buffer (pH 7.15), and the cells are maintained in air enriched with CO₂ (0.8%). The cells are kept continuously lit under 100 μE/m²/sec. for 7 days. Replica plates are generated containing each independent transformant.

ATP Assay: ATP levels in cells are measured using the Promega ENLITEN® ATP Assay (Promega Inc., Madison, Wis.). Plates containing cells are spun in a swinging bucket centrifuge at 10,000×g for 15 minutes and excess cell media is removed. Cells are extracted with trichloroacetic acid (TCA) according to the manufacturer's instructions and acidity of the sample is neutralized. The cell material in each well is then subjected to ATP assay using the Promega ENLITEN® ATP Assay according to the manufacturer's instructions. Plates are analyzed by a Veritas™ Microplate Luminometer (Promega Inc., Madison. Wis.). Strains that generate a higher luciferase signal than the starting strain are selected for further development from replica plates.

1-42. (canceled)

43. A photosynthetic microorganism containing an antisense or RNAi construct that targets a transcript of a gene that encodes a protein involved in light harvesting, wherein the antisense or RNAi construct is in operable linkage with a promoter.

44-45. (canceled)

46. A population of photosynthetic microorganisms in liquid culture media, wherein:
a. the population is exposed to light from above the plane of the surface of the culture media;
b. at least one cell in the population contains an antisense or RNAi segment comprising at least 10 nucleotides of a gene encoding a protein involved in light harvesting in operable linkage with a promoter; and
c. cells on the top of the population express the antisense or RNAi segment at a higher level than cells on the bottom of the population.

47. The population of claim 46, wherein the cells of the population are incapable of flagella-based motility.

48-51. (canceled)

52. The photosynthetic microorganism of claim 43, wherein the microorganism contains an RNAi construct that encodes an RNAi molecule.

53. The photosynthetic microorganism of claim 52, wherein the RNAi molecule targets transcripts from more than one gene.

54. The photosynthetic microorganism of claim 43, wherein the microorganism contains an antisense construct that encodes an antisense molecule.

55. The photosynthetic microorganism of claim 54, wherein the antisense molecule targets transcripts from more than one gene.

56. (canceled)

57. The photosynthetic microorganism of claim 52, wherein the RNAi molecule targets transcripts from only one gene.

58. The photosynthetic microorganism of claim 54, wherein the antisense molecule targets transcripts from only one gene.

59. The photosynthetic microorganism of claim 43, wherein the antisense or RNAi construct comprises at least 10 nucleotides targeting a gene that encodes a protein that binds at least one light absorbing pigment.

60. The photosynthetic microorganism of claim 43, wherein the antisense or RNAi construct comprises at least 10 nucleotides targeting a gene that encodes a protein that catalyzes biosynthetic production of light absorbing pigment molecules.

61. The photosynthetic microorganism of claim 43, wherein the antisense or RNAi construct comprises at least 10 nucleotides targeting a gene that encodes a protein that modulates photosynthetic activity through signal transduction.

62. The photosynthetic microorganism of claim 43, wherein the antisense or RNAi construct comprises at least 10 nucleotides targeting a gene that encodes a protein that dissipates absorbed light energy as heat.

63. The photosynthetic microorganism of claim 43, wherein the antisense or RNAi construct comprises at least 10 nucleotides targeting a gene that encodes a protein listed in Table 1.

64. The photosynthetic microorganism of claim 43, wherein the antisense or RNAi construct comprises at least 10 nucleotides targeting a gene that encodes a protein that has at least 50% amino acid sequence identity with a protein listed in Table 1.

65. The photosynthetic microorganism of claim 43, wherein the antisense or RNAi construct comprises at least 10 nucleotides targeting a gene that encodes a light harvesting antenna protein.

66. The photosynthetic microorganism of claim 43, wherein the microorganism has a higher level of oxygen evolution than that of a starting strain.

67. The photosynthetic microorganism of claim 43, wherein the microorganism has a higher level of ATP production than that of a starting strain.

68. The photosynthetic microorganism of claim 43, wherein the microorganism has a higher level of hydrogen production than that of a starting strain.

69. The photosynthetic microorganism of claim 43, wherein the microorganism has a higher level of production of a recombinant protein than that of a starting strain.

70. The photosynthetic microorganism of claim 43, wherein the microorganism has a higher level of production of an isoprenoid than that of a starting strain.

71. The photosynthetic microorganism of claim 73, wherein the microorganism has a higher level of production of a carotenoid than that of a starting strain.

72. The photosynthetic microorganism of claim 43, wherein the microorganism has a higher level of production of a lipid than that of a starting strain.

73. The photosynthetic microorganism of claim 43, wherein the microorganism has a higher level of production of a hydrocolloid than that of a starting strain.

74. The photosynthetic microorganism of claim 43, wherein the microorganism has a higher level of production of a polyketoid than that of a starting strain.

75. The photosynthetic microorganism of claim 43, wherein the microorganism has a higher level of production of a fatty acid than that of a starting strain.

76. The photosynthetic microorganism of claim 43, wherein the microorganism has a higher level of production of a polysaccharide than that of a starting strain.

77. The photosynthetic microorganism of claim 43, wherein the microorganism has a higher level of production of an antibiotic than that of a starting strain.

78. The photosynthetic microorganism of claim 43, further comprising at least one heterologous gene encoding an enzyme that participates in the synthesis of a molecule from the list consisting of a hydrocolloid, isoprenoid, polyketoid, fatty acid, lipid, carotenoid, polysaccharide, or antibiotic molecule.

79. The photosynthetic microorganism of claim 43, further comprising at least one heterologous gene encoding a recombinant human protein selected from the list consisting of insulin, interferon alpha, erythropoietin, human growth hormone, granulocyte-colony stimulating factor, tissue plasminogen activator, a human immumoglobulin and Factor VIII.

80. The photosynthetic microorganism of claim 43, wherein the microorganism is eukaryotic.

81. The photosynthetic microorganism of claim 80, wherein the microorganism is of a genus selected from the group consisting of Chlamydomonas, Chlorella, Volvox, Phaeodactylum, Dunaliella and Thalassiosira.

82. The photosynthetic microorganism of claim 81, wherein the microorganism is selected from the group consisting of Chlorella fusca, Chlorella protothecoides, Chlorella pyrenoidosa, Chlorella kessleri, Chlorella vulgaris and Chlorella ellipsoidea.

83. The photosynthetic microorganism of claim 81, wherein the microorganism is Dunaliella salina or Dunaliella bardawil.

84. The photosynthetic microorganism of claim 43, wherein the microorganism is prokaryotic.

85. The photosynthetic microorganism of claim 84, wherein the microorganism is of a genus selected from the group consisting of Thermosynechococcus, Synechococcus, Anabaena, Synechocystis, Nephroselmis, Trichodesmium and Fremyella.

86. The photosynthetic microorganism of claim 43, wherein the microorganism is listed in Table 4.

87. (canceled)

88. The photosynthetic microorganism of claim 43, wherein the microorganism exhibits a lower level of heat dissipation than a starting strain.

89. The photosynthetic microorganism of claim 43, further comprising at least one heterologous gene encoding an enzyme that participates in the synthesis of a lipid.

90. The microorganism of claim 43, wherein the promoter is activated by light.

91. The population of claim 46, wherein the promoter is activated by light.


SEQUENCE LISTING (62 sequences)

SEQ ID NO: 1 - DNA, 2528 bp, Chlamydomonas reinhardtii
ttggattgcacagtttctaacaggtgatatgctatttaagatacttacagtaaataactggcaatggacctatcgcatgtacagctctcgttggcgtgaagtttgctagtcgcgagagagcaggccacgaggcggggagtttaggggtatcgcatgcgaatggctgcttggcgcttttaaacaagtatatccatgtgaagaatgcagatggggcgagtatgcaggccgggggcctgggagcatgctccgtgattgcgcacgggcgatgagccgatgacgcatgcatcacggacgggttgtcatgcgcgggcgtggtttttgcacaggcatgtgtgattttgcgtgtgccgcgtggatggctgcaagcagcagcctgtgaggaacaggaagcattcgacgcgtgcaggggcacgcgcacagcagcagacacagcagccgcggcagacacagcagccgcgggcatgcaccctgctcatcccccacacgccgttcccgcgtcgactctattgcttccaggccccgctaagttgctcgggatccaagtgcagtgcatgccagctacgaccgaaccattctgtgacttgtgttctgtggtgaacttactactgccgtggtgcagctattaggccgtgtgcagggccgccggggtggccagcttggccctcaggccctggcctcgtgagtcttgtgacctgccttgaactggcacgccgctgatgtctacagcagggctttcgtactattagtgcgtgtgggctgcgtcagcaggtccctcgtgtaacccatgcaacatagtcagtatctcatgtgctgggttgcagttgcattgtgtaaggttgcgcctctgtcaagccccatcactccggggtgtctgtatcaggctagcaccgtagctaacctcatctcgccccagggccccttggtgccagggatgcaccccctgaaaggtagtgtgtgaggcgcggcagcaaaatccctccacgttacaccgccttcaccactctgcatgcctggcacgaccacacgcgcgcccaggcactgggcaaacggcgcgtccgggtggtgcgctcggtacatccgagggccgcattcgcccgcctgaccatccatataacttgggccgatacgcagagtccatagcagtcatgcgagcctcaactcactcggttcacatccgcctcacacatccgccgcggcccaactccaacccgggtccctgtgtcgcaatcaacctggcactatctgtgagtgtctctgcaccggtaatcggtaggcatctagctgccggcaacttgtcaggccagacgtaagtctcatgttcttctctgcctcctcataccaagaagggaccgagcataactgcccgttgaccctctaagcgtacacagacgcattcgagcaacacacgtcactcaatagctgcactgcgctcaagcgcatgtgccagacaccgtcgggcaacgcgcgcgccagcagccgtcattcgactcctcaactcccctcaaccgctctgatccatacgctctgctttgtggagtagcactagaatatctatttagcacggccgcagccgcgcccttaagctcccactcctaacacacgcgcacgcatacgttcacccatgccccaaacccgggctcaagccacagcaccaccaccactgcttctgtaagccaccaccgcacacttacacaccatctacttctgcagctccgcccaacccttccccatggtcggctccggtcgtgagctgaacttgccggtggcgcccttccactcgcccacgcctagcgagtagccccagtcggattgggtctccagcgtgaggtcggacgtggcgcgcgtcatgcacagcagcgccatgcccttggcctgctcctcctcgtctagcgtgaaggtgaggtcagcgatgtcggaggggtcgatggttcccttggccacgcgcgccacgcaggcgccgcagatgccgccgcggcaggtggcgggcaggtccaggccctgcgcctccgcggcatcgaggatgtattggttgtccgggcaggaaatctcgcgagtctcgccgtcggcaccgacgaaggtgaccttgtagctgatgcccttcgactccacaccgcgtacggctgaaaccttgtcgcggcagaaagcgtcggaggcatgcacacgcacgctgcgcgcgcggctgcccatgaccgggcgcactgcagacggcgcggatgtgctggctagggtgctgctacgcagcgaggcagtcatcgaggccattcctacagagtaaaggtctaggcgatgcgcgactgaaagactgtgaatcccggcgtcgccgtggtgggatgtgggccggtgcgctgtcgcagaggataaattacaggtatcaaacaaggttagggcgttggaaggagcggcgctagggaactgaaatcggatctgcatcggaccctcattccgcgacttgtccttcttttgcctcgccccgcagctcttgagttttgttcttgaccctttgacacgaaccaaccgatataaaaatg

SEQ ID NO: 2 - DNA, 2525 bp, Chlamydomonas reinhardtii
ttggattgcacagtttctaacaggtgatatgctatttaagatacttacagtaaataactggcaatggacctatcgcatgtacagctctcgttggcgtgaagtttgctagtcgcgagagagcaggccacgaggcggggagtttaggggtatcgcatgcgaatggctgcttggcgcttttaaacaagtatatccatgtgaagaatgcagatggggcgagtatgcaggccgggggcctgggagcatgctccgtgattgcgcacgggcgatgagccgatgacgcatgcatcacggacgggttgtcatgcgcgggcgtggtttttgcacaggcatgtgtgattttgcgtgtgccgcgtggatggctgcaagcagcagcctgtgaggaacaggaagcattcgacgcgtgcaggggcacgcgcacagcagcagacacagcagccgcggcagacacagcagccgcgggcatgcaccctgctcatcccccacacgccgttcccgcgtcgactctattgcttccaggccccgctaagttgctcgggatccaagtgcagtgcatgccagctacgaccgaaccattctgtgacttgtgttctgtggtgaacttactactgccgtggtgcagctattaggccgtgtgcagggccgccggggtggccagcttggccctcaggccctggcctcgtgagtcttgtgacctgccttgaactggcacgccgctgatgtctacagcagggctttcgtactattagtgcgtgtgggctgcgtcagcaggtccctcgtgtaacccatgcaacatagtcagtatctcatgtgctgggttgcagttgcattgtgtaaggttgcgcctctgtcaagccccatcactccggggtgtctgtatcaggctagcaccgtagctaacctcatctcgccccagggccccttggtgccagggatgcaccccctgaaaggtagtgtgtgaggcgcggcagcaaaatccctccacgttacaccgccttcaccactctgcatgcctggcacgaccacacgcgcgcccaggcactgggcaaacggcgcgtccgggtggtgcgctcggtacatccgagggccgcattcgcccgcctgaccatccatataacttgggccgatacgcagagtccatagcagtcatgcgagcctcaactcactcggttcacatccgcctcacacatccgccgcggcccaactccaacccgggtccctgtgtcgcaatcaacctggcactatctgtgagtgtctctgcaccggtaatcggtaggcatctagctgccggcaacttgtcaggccagacgtaagtctcatgttcttctctgcctcctcataccaagaagggaccgagcataactgcccgttgaccctctaagcgtacacagacgcattcgagcaacacacgtcactcaatagctgcactgcgctcaagcgcatgtgccagacaccgtcgggcaacgcgcgcgccagcagccgtcattcgactcctcaactcccctcaaccgctctgatccatacgctctgctttgtggagtagcactagaatatctatttagcacggccgcagccgcgcccttaagctcccactcctaacacacgcgcacgcatacgttcacccatgccccaaacccgggctcaagccacagcaccaccaccactgcttctgtaagccaccaccgcacacttacacaccatctacttctgcagctccgcccaacccttccccatggtcggctccggtcgtgagctgaacttgccggtggcgcccttccactcgcccacgcctagcgagtagccccagtcggattgggtctccagcgtgaggtcggacgtggcgcgcgtcatgcacagcagcgccatgcccttggcctgctcctcctcgtctagcgtgaaggtgaggtcagcgatgtcggaggggtcgatggttcccttggccacgcgcgccacgcaggcgccgcagatgccgccgcggcaggtggcgggcaggtccaggccctgcgcctccgcggcatcgaggatgtattggttgtccgggcaggaaatctcgcgagtctcgccgtcggcaccgacgaaggtgaccttgtagctgatgcccttcgactccacaccgcgtacggctgaaaccttgtcgcggcagaaagcgtcggaggcatgcacacgcacgctgcgcgcgcggctgcccatgaccgggcgcactgcagacggcgcggatgtgctggctagggtgctgctacgcagcgaggcagtcatcgaggccattcctacagagtaaaggtctaggcgatgcgcgactgaaagactgtgaatcccggcgtcgccgtggtgggatgtgggccggtgcgctgtcgcagaggataaattacaggtatcaaacaaggttagggcgttggaaggagcggcgctagggaactgaaatcggatctgcatcggaccctcattccgcgacttgtccttcttttgcctcgccccgcagctcttgagttttgttcttgaccctttgacacgaaccaaccgatataaaa

SEQ ID NO: 3 - DNA, 2110 bp, Chlamydomonas reinhardtii
cacagcagcagacacagcagccgcggcagacacagcagccgcgggcatgcaccctgctcatcccccacacgccgttcccgcgtcgactctattgcttccaggccccgctaagttgctcgggatccaagtgcagtgcatgccagctacgaccgaaccattctgtgacttgtgttctgtggtgaacttactactgccgtggtgcagctattaggccgtgtgcagggccgccggggtggccagcttggccctcaggccctggcctcgtgagtcttgtgacctgccttgaactggcacgccgctgatgtctacagcagggctttcgtactattagtgcgtgtgggctgcgtcagcaggtccctcgtgtaacccatgcaacatagtcagtatctcatgtgctgggttgcagttgcattgtgtaaggttgcgcctctgtcaagccccatcactccggggtgtctgtatcaggctagcaccgtagctaacctcatctcgccccagggccccttggtgccagggatgcaccccctgaaaggtagtgtgtgaggcgcggcagcaaaatccctccacgttacaccgccttcaccactctgcatgcctggcacgaccacacgcgcgcccaggcactgggcaaacggcgcgtccgggtggtgcgctcggtacatccgagggccgcattcgcccgcctgaccatccatataacttgggccgatacgcagagtccatagcagtcatgcgagcctcaactcactcggttcacatccgcctcacacatccgccgcggcccaactccaacccgggtccctgtgtcgcaatcaacctggcactatctgtgagtgtctctgcaccggtaatcggtaggcatctagctgccggcaacttgtcaggccagacgtaagtctcatgttcttctctgcctcctcataccaagaagggaccgagcataactgcccgttgaccctctaagcgtacacagacgcattcgagcaacacacgtcactcaatagctgcactgcgctcaagcgcatgtgccagacaccgtcgggcaacgcgcgcgccagcagccgtcattcgactcctcaactcccctcaaccgctctgatccatacgctctgctttgtggagtagcactagaatatctatttagcacggccgcagccgcgcccttaagctcccactcctaacacacgcgcacgcatacgttcacccatgccccaaacccgggctcaagccacagcaccaccaccactgcttctgtaagccaccaccgcacacttacacaccatctacttctgcagctccgcccaacccttccccatggtcggctccggtcgtgagctgaacttgccggtggcgcccttccactcgcccacgcctagcgagtagccccagtcggattgggtctccagcgtgaggtcggacgtggcgcgcgtcatgcacagcagcgccatgcccttggcctgctcctcctcgtctagcgtgaaggtgaggtcagcgatgtcggaggggtcgatggttcccttggccacgcgcgccacgcaggcgccgcagatgccgccgcggcaggtggcgggcaggtccaggccctgcgcctccgcggcatcgaggatgtattggttgtccgggcaggaaatctcgcgagtctcgccgtcggcaccgacgaaggtgaccttgtagctgatgcccttcgactccacaccgcgtacggctgaaaccttgtcgcggcagaaagcgtcggaggcatgcacacgcacgctgcgcgcgcggctgcccatgaccgggcgcactgcagacggcgcggatgtgctggctagggtgctgctacgcagcgaggcagtcatcgaggccattcctacagagtaaaggtctaggcgatgcgcgactgaaagactgtgaatcccggcgtcgccgtggtgggatgtgggccggtgcgctgtcgcagaggataaattacaggtatcaaacaaggttagggcgttggaaggagcggcgctagggaactgaaatcggatctgcatcggaccctcattccgcgacttgtccttcttttgcctcgccccgcagctcttgagttttgttcttgaccctttgacacgaaccaaccgatataaaa

SEQ ID NO: 4 - DNA, 1679 bp, Chlamydomonas reinhardtii
gtcaagccccatcactccggggtgtctgtatcaggctagcaccgtagctaacctcatctcgccccagggccccttggtgccagggatgcaccccctgaaaggtagtgtgtgaggcgcggcagcaaaatccctccacgttacaccgccttcaccactctgcatgcctggcacgaccacacgcgcgcccaggcactgggcaaacggcgcgtccgggtggtgcgctcggtacatccgagggccgcattcgcccgcctgaccatccatataacttgggccgatacgcagagtccatagcagtcatgcgagcctcaactcactcggttcacatccgcctcacacatccgccgcggcccaactccaacccgggtccctgtgtcgcaatcaacctggcactatctgtgagtgtctctgcaccggtaatcggtaggcatctagctgccggcaacttgtcaggccagacgtaagtctcatgttcttctctgcctcctcataccaagaagggaccgagcataactgcccgttgaccctctaagcgtacacagacgcattcgagcaacacacgtcactcaatagctgcactgcgctcaagcgcatgtgccagacaccgtcgggcaacgcgcgcgccagcagccgtcattcgactcctcaactcccctcaaccgctctgatccatacgctctgctttgtggagtagcactagaatatctatttagcacggccgcagccgcgcccttaagctcccactcctaacacacgcgcacgcatacgttcacccatgccccaaacccgggctcaagccacagcaccaccaccactgcttctgtaagccaccaccgcacacttacacaccatctacttctgcagctccgcccaacccttccccatggtcggctccggtcgtgagctgaacttgccggtggcgcccttccactcgcccacgcctagcgagtagccccagtcggattgggtctccagcgtgaggtcggacgtggcgcgcgtcatgcacagcagcgccatgcccttggcctgctcctcctcgtctagcgtgaaggtgaggtcagcgatgtcggaggggtcgatggttcccttggccacgcgcgccacgcaggcgccgcagatgccgccgcggcaggtggcgggcaggtccaggccctgcgcctccgcggcatcgaggatgtattggttgtccgggcaggaaatctcgcgagtctcgccgtcggcaccgacgaaggtgaccttgtagctgatgcccttcgactccacaccgcgtacggctgaaaccttgtcgcggcagaaagcgtcggaggcatgcacacgcacgctgcgcgcgcggctgcccatgaccgggcgcactgcagacggcgcggatgtgctggctagggtgctgctacgcagcgaggcagtcatcgaggccattcctacagagtaaaggtctaggcgatgcgcgactgaaagactgtgaatcccggcgtcgccgtggtgggatgtgggccggtgcgctgtcgcagaggataaattacaggtatcaaacaaggttagggcgttggaaggagcggcgctagggaactgaaatcggatctgcatcggaccctcattccgcgacttgtccttcttttgcctcgccccgcagctcttgagttttgttcttgaccctttgacacgaaccaaccgatataaaa

SEQ ID NO: 5 - DNA, 1938 bp, Chlamydomonas reinhardtii
ttggattgcacagtttctaacaggtgatatgctatttaagatacttacagtaaataactggcaatggacctatcgcatgtacagctctcgttggcgtgaagtttgctagtcgcgagagagcaggccacgaggcggggagtttaggggtatcgcatgcgaatggctgcttggcgcttttaaacaagtatatccatgtgaagaatgcagatggggcgagtatgcaggccgggggcctgggagcatgctccgtgattgcgcacgggcgatgagccgatgacgcatgcatcacggacgggttgtcatgcgcgggcgtggtttttgcacaggcatgtgtgattttgcgtgtgccgcgtggatggctgcaagcagcagcctgtgaggaacaggaagcattcgacgcgtgcaggggcacgcgcacagcagcagacacagcagccgcggcagacacagcagccgcgggcatgcaccctgctcatcccccacacgccgttcccgcgtcgactctattgcttccaggccccgctaagttgctcgggatccaagtgcagtgcatgccagctacgaccgaaccattctgtgacttgtgttctgtggtgaacttactactgccgtggtgcagctattaggccgtgtgcagggccgccggggtggccagcttggccctcaggccctggcctcgtgagtcttgtgacctgccttgaactggcacgccgctgatgtctacagcagggctttcgtactattagtgcgtgtgggctgcgtcagcaggtccctcgtgtaacccatgcaacatagtcagtatctcatgtgctgggttgcagttgcattgtgtaaggttgcgcctctgtcaagccccatcactccggggtgtctgtatcaggctagcaccgtagctaacctcatctcgccccagggccccttggtgccagggatgcaccccctgaaaggtagtgtgtgaggcgcggcagcaaaatccctccacgttacaccgccttcaccactctgcatgcctggcacgaccacacgcgcgcccaggcactgggcaaacggcgcgtccgggtggtgcgctcggtacatccgagggccgcattcgcccgcctgaccatccatataacttgggccgatacgcagagtccatagcagtcatgcgagcctcaactcactcggttcacatccgcctcacacatccgccgcggcccaactccaacccgggtccctgtgtcgcaatcaacctggcactatctgtgagtgtctctgcaccggtaatcggtaggcatctagctgccggcaacttgtcaggccagacgtaagtctcatgttcttctctgcctcctcataccaagaagggaccgagcataactgcccgttgaccctctaagcgtacacagacgcattcgagcaacacacgtcactcaatagctgcactgcgctcaagcgcatgtgccagacaccgtcgggcaacgcgcgcgccagcagccgtcattcgactcctcaactcccctcaaccgctctgatccatacgctctgctttgtggagtagcactagaatatctatttagcacggccgcagccgcgcccttaagctcccactcctaacacacgcgcacgcatacgttcacccatgccccaaacccgggctcaagccacagcaccaccaccactgcttctgtaagccaccaccgcacacttacacaccatctacttctgcagctccgcccaacccttccccatggtcggctccggtcgtgagctgaacttgccggtggcgcccttccactcgcccacgcctagcgagtagccccagtcggattgggtctccagcgtgaggtcggacgtggcgcgcgtcatgcacagcagcgccatgcccttggcctgctcctcctcgtctagcgtgaaggtgaggtcagcgatgtcggaggggtcgatggttcccttggc

SEQ ID NO: 6 - DNA, 1523 bp, Chlamydomonas reinhardtii
cacagcagcagacacagcagccgcggcagacacagcagccgcgggcatgcaccctgctcatcccccacacgccgttcccgcgtcgactctattgcttccaggccccgctaagttgctcgggatccaagtgcagtgcatgccagctacgaccgaaccattctgtgacttgtgttctgtggtgaacttactactgccgtggtgcagctattaggccgtgtgcagggccgccggggtggccagcttggccctcaggccctggcctcgtgagtcttgtgacctgccttgaactggcacgccgctgatgtctacagcagggctttcgtactattagtgcgtgtgggctgcgtcagcaggtccctcgtgtaacccatgcaacatagtcagtatctcatgtgctgggttgcagttgcattgtgtaaggttgcgcctctgtcaagccccatcactccggggtgtctgtatcaggctagcaccgtagctaacctcatctcgccccagggccccttggtgccagggatgcaccccctgaaaggtagtgtgtgaggcgcggcagcaaaatccctccacgttacaccgccttcaccactctgcatgcctggcacgaccacacgcgcgcccaggcactgggcaaacggcgcgtccgggtggtgcgctcggtacatccgagggccgcattcgcccgcctgaccatccatataacttgggccgatacgcagagtccatagcagtcatgcgagcctcaactcactcggttcacatccgcctcacacatccgccgcggcccaactccaacccgggtccctgtgtcgcaatcaacctggcactatctgtgagtgtctctgcaccggtaatcggtaggcatctagctgccggcaacttgtcaggccagacgtaagtctcatgttcttctctgcctcctcataccaagaagggaccgagcataactgcccgttgaccctctaagcgtacacagacgcattcgagcaacacacgtcactcaatagctgcactgcgctcaagcgcatgtgccagacaccgtcgggcaacgcgcgcgccagcagccgtcattcgactcctcaactcccctcaaccgctctgatccatacgctctgctttgtggagtagcactagaatatctatttagcacggccgcagccgcgcccttaagctcccactcctaacacacgcgcacgcatacgttcacccatgccccaaacccgggctcaagccacagcaccaccaccactgcttctgtaagccaccaccgcacacttacacaccatctacttctgcagctccgcccaacccttccccatggtcggctccggtcgtgagctgaacttgccggtggcgcccttccactcgcccacgcctagcgagtagccccagtcggattgggtctccagcgtgaggtcggacgtggcgcgcgtcatgcacagcagcgccatgcccttggcctgctcctcctcgtctagcgtgaaggtgaggtcagcgatgtcggaggggtcgatggttcccttggc

SEQ ID NO: 7 - DNA, 1092 bp, Chlamydomonas reinhardtii
gtcaagccccatcactccggggtgtctgtatcaggctagcaccgtagctaacctcatctcgccccagggccccttggtgccagggatgcaccccctgaaaggtagtgtgtgaggcgcggcagcaaaatccctccacgttacaccgccttcaccactctgcatgcctggcacgaccacacgcgcgcccaggcactgggcaaacggcgcgtccgggtggtgcgctcggtacatccgagggccgcattcgcccgcctgaccatccatataacttgggccgatacgcagagtccatagcagtcatgcgagcctcaactcactcggttcacatccgcctcacacatccgccgcggcccaactccaacccgggtccctgtgtcgcaatcaacctggcactatctgtgagtgtctctgcaccggtaatcggtaggcatctagctgccggcaacttgtcaggccagacgtaagtctcatgttcttctctgcctcctcataccaagaagggaccgagcataactgcccgttgaccctctaagcgtacacagacgcattcgagcaacacacgtcactcaatagctgcactgcgctcaagcgcatgtgccagacaccgtcgggcaacgcgcgcgccagcagccgtcattcgactcctcaactcccctcaaccgctctgatccatacgctctgctttgtggagtagcactagaatatctatttagcacggccgcagccgcgcccttaagctcccactcctaacacacgcgcacgcatacgttcacccatgccccaaacccgggctcaagccacagcaccaccaccactgcttctgtaagccaccaccgcacacttacacaccatctacttctgcagctccgcccaacccttccccatggtcggctccggtcgtgagctgaacttgccggtggcgcccttccactcgcccacgcctagcgagtagccccagtcggattgggtctccagcgtgaggtcggacgtggcgcgcgtcatgcacagcagcgccatgcccttggcctgctcctcctcgtctagcgtgaaggtgaggtcagcgatgtcggaggggtcgatggttcccttggc

SEQ ID NO: 8 - DNA, 1165 bp, Chlamydomonas reinhardtii
ttggattgcacagtttctaacaggtgatatgctatttaagatacttacagtaaataactggcaatggacctatcgcatgtacagctctcgttggcgtgaagtttgctagtcgcgagagagcaggccacgaggcggggagtttaggggtatcgcatgcgaatggctgcttggcgcttttaaacaagtatatccatgtgaagaatgcagatggggcgagtatgcaggccgggggcctgggagcatgctccgtgattgcgcacgggcgatgagccgatgacgcatgcatcacggacgggttgtcatgcgcgggcgtggtttttgcacaggcatgtgtgattttgcgtgtgccgcgtggatggctgcaagcagcagcctgtgaggaacaggaagcattcgacgcgtgcaggggcacgcgcacagcagcagacacagcagccgcggcagacacagcagccgcgggcatgcaccctgctcatcccccacacgccgttcccgcgtcgactctattgcttccaggccccgctaagttgctcgggatccaagtgcagtgcatgccagctacgaccgaaccattctgtgacttgtgttctgtggtgaacttactactgccgtggtgcagctattaggccgtgtgcagggccgccggggtggccagcttggccctcaggccctggcctcgtgagtcttgtgacctgccttgaactggcacgccgctgatgtctacagcagggctttcgtactattagtgcgtgtgggctgcgtcagcaggtccctcgtgtaacccatgcaacatagtcagtatctcatgtgctgggttgcagttgcattgtgtaaggttgcgcctctgtcaagccccatcactccggggtgtctgtatcaggctagcaccgtagctaacctcatctcgccccagggccccttggtgccagggatgcaccccctgaaaggtagtgtgtgaggcgcggcagcaaaatccctccacgttacaccgccttcaccactctgcatgcctggcacgaccacacgcgcgcccaggcactgggcaaacggcgcgtccgggtggtgcgctcggtacatccgagggccgcattcgcccgcctgaccatccatataacttgggccgatacgcagagtccatagcagtcatgcgagcctcaactcactc

SEQ ID NO: 9 - DNA, 750 bp, Chlamydomonas reinhardtii
cacagcagcagacacagcagccgcggcagacacagcagccgcgggcatgcaccctgctcatcccccacacgccgttcccgcgtcgactctattgcttccaggccccgctaagttgctcgggatccaagtgcagtgcatgccagctacgaccgaaccattctgtgacttgtgttctgtggtgaacttactactgccgtggtgcagctattaggccgtgtgcagggccgccggggtggccagcttggccctcaggccctggcctcgtgagtcttgtgacctgccttgaactggcacgccgctgatgtctacagcagggctttcgtactattagtgcgtgtgggctgcgtcagcaggtccctcgtgtaacccatgcaacatagtcagtatctcatgtgctgggttgcagttgcattgtgtaaggttgcgcctctgtcaagccccatcactccggggtgtctgtatcaggctagcaccgtagctaacctcatctcgccccagggccccttggtgccagggatgcaccccctgaaaggtagtgtgtgaggcgcggcagcaaaatccctccacgttacaccgccttcaccactctgcatgcctggcacgaccacacgcgcgcccaggcactgggcaaacggcgcgtccgggtggtgcgctcggtacatccgagggccgcattcgcccgcctgaccatccatataacttgggccgatacgcagagtccatagcagtcatgcgagcctcaactcactc

SEQ ID NO: 10 - DNA, 319 bp, Chlamydomonas reinhardtii
gtcaagccccatcactccggggtgtctgtatcaggctagcaccgtagctaacctcatctcgccccagggccccttggtgccagggatgcaccccctgaaaggtagtgtgtgaggcgcggcagcaaaatccctccacgttacaccgccttcaccactctgcatgcctggcacgaccacacgcgcgcccaggcactgggcaaacggcgcgtccgggtggtgcgctcggtacatccgagggccgcattcgcccgcctgaccatccatataacttgggccgatacgcagagtccatagcagtcatgcgagcctcaactcactc

SEQ ID NO: 11 - DNA, 51 bp, artificial sequence
  synthetic construct
ttagtaaagatacaatgattcatcaatttggattgcacagtttctaacagg

SEQ ID NO: 12 - DNA, 48 bp, artificial sequence
  synthetic construct
ttagtaaagatacaatgattcatcaatcacagcagcagacacagcagc

SEQ ID NO: 13 - DNA, 47 bp, artificial sequence
  synthetic construct
ttagtaaagatacaatgattcatcaatgtcaagccccatcactccgg

SEQ ID NO: 14 - DNA, 48 bp, artificial sequence
  synthetic construct
aagtacttaaatcatctaatcgttcttgagtgagttgaggctcgcatg

SEQ ID NO: 15 - DNA, 47 bp, artificial sequence
  synthetic construct
aagtacttaaatcatctaatcgttcttgccaagggaaccatcgaccc

SEQ ID NO: 16 - DNA, 50 bp, artificial sequence
  synthetic construct
aagtacttaaatcatctaatcgttcttttttatatcggttggttcgtgtc

SEQ ID NO: 17 - DNA, 27 bp, artificial sequence
  synthetic construct
ttagtaaagatacaatgattcatcaat

SEQ ID NO: 18 - DNA, 27 bp, artificial sequence
  synthetic construct
aagtacttaaatcatctaatcgttctt

SEQ ID NO: 19 - DNA, 24 bp, artificial sequence
  synthetic construct
atactcatcgatagctatcgtaga

SEQ ID NO: 20 - DNA, 24 bp, artificial sequence
  synthetic construct
tctacgatagctatcgatgagtat

SEQ ID NO: 21 - DNA, 83 bp, artificial sequence
  synthetic construct
atactcatcgatagctatcgtagaaaggaggtcaagaacggccgcctggtaaatcgatccaggcggccgttcttgacctcctt

SEQ ID NO: 22 - DNA, 25 bp, artificial sequence
  synthetic construct
aaggaggtcaagaacggccgcctgg

SEQ ID NO: 23 - DNA, 9 bp, artificial sequence
  synthetic construct
taaatcgat

SEQ ID NO: 24 - DNA, 25 bp, artificial sequence
  synthetic construct
ccaggcggccgttcttgacctcctt

SEQ ID NO: 25 - DNA, 24 bp, artificial sequence
  synthetic construct
ggctgggcccctactctgagaacg

SEQ ID NO: 26 - DNA, 24 bp, artificial sequence
  synthetic construct
cgttctcagagtaggggcccagcc

SEQ ID NO: 27 - DNA, 22 bp, artificial sequence
  synthetic construct
tcgcccaggtggagtcctacac

SEQ ID NO: 28 - DNA, 21 bp, artificial sequence
  synthetic construct
tgtaggactccacctgggcga

SEQ ID NO: 29 - DNA, 21 bp, artificial sequence
  synthetic construct
gtggtgtcatgatcatgggcg

SEQ ID NO: 30 - DNA, 21 bp, artificial sequence
  synthetic construct
cgcccatgatcatgacaccac

SEQ ID NO: 31 - DNA, 23 bp, artificial sequence
  synthetic construct
tacctgactggcgagttccccgg

SEQ ID NO: 32 - DNA, 23 bp, artificial sequence
  synthetic construct
ccggggaactcgccagtcaggta

SEQ ID NO: 33 - DNA, 24 bp, artificial sequence
  synthetic construct
gcagatcggccagggcttctggga

SEQ ID NO: 34 - DNA, 25 bp, artificial sequence
  synthetic construct
tcaagaacggccgcctggccatggt

SEQ ID NO: 35 - DNA, 25 bp, artificial sequence
  synthetic construct
gactacggctgggacaccgccggtc

SEQ ID NO: 36 - DNA, 21 bp, artificial sequence
  synthetic construct
gagctgaagaccctgcagacc

SEQ ID NO: 37 - DNA, 21 bp, artificial sequence
  synthetic construct
gagctcaaggtcatgcagacc

SEQ ID NO: 38 - DNA, 223 bp, Synechococcus PCC7942
gtcgacacatctatgacttggccttgatgacccaaaaagggtttgatgctgagggaatgaaagccttcattgagcgttctaatgcggtcttgacggcgttgacgactcgccagtgaagggtttaggggaactctaaacttcacagaacgtcatcctagctatctcgccctcagacgtgagcctcgctaagctgagggcgacgatccatttcgtggtgtggggt

SEQ ID NO: 39 - DNA, 525 bp, Synechococcus sp. WH8102
atgcgcgacgccatcggcggactgatcggccgttacgaccaattgggtcggtatctggaccgctcggcgatcgacagcatcgaaaagtacctcgatgaatcgtccctgcggatccaggccgtggagctcatcaaccgggaagcggctgaaatcgtgcgggaggcaagtcagcggctgttccgtgatgagccggaacttctcctccctggtgggaatgcctacaccaccagacgcctcgcggcctgtctgcgggatatggactatttcctccgctatgccagttacgcactggttgcggcagacagcacaattttgaacgaaagggtgctcaacggtctggacgacacctacaaaagcttgggcgtgcccacggggccaaccgttcgcagcatcatcctcttgggtgaagtgatcgttgagcgccttcaggccgcaggagttgaatcagctcggctggcggttgttgctgcaccctttgatcacatggcccgtggtcttgctgaaacgaatgttcggcagcgctga

SEQ ID NO: 40 - DNA, 89 bp, artificial sequence
  synthetic construct
gaggaaatagtccatatcccgcagacaggccgcgaggcgtctggtggtgtaggcattcccaccagggaggagaagttccggctcatcac

SEQ ID NO: 41 - DNA, 1389 bp, Synechococcus sp. WH8102
gtggtaacgctctctaatcccggtcttggcgccactggcggcaaagacctcgactccaccggctacgcctggtggtctggcaacgctcgtctgatcaacctgtctggccgtctgctcggcgcccacgtggcgcacgctggcctgatggtgttctgggccggcgccatgatgctgttcgaggtgagtcacttcaccttcgacaaacccatgtacgaacagggcttcatctgcatgccccacgtcgccacccttggctacggcgtgggccccggcggtgaggtcactgatctcttccccttcttcgtggtcggtgttctgcacctgatcagctctgccgttctcggcctgggcggtctgtaccatgctctgcgcggtcctgagattctggagaactactcttccttcttctcccaggactggcgcgacaagaaccagatgaccaacatcatcggttatcacttgatccttctgggcgtcggctgcctgctgctggtcttcaaggccatgttcttcggtggcgtctacgacacctgggcccccggcggcggtgacgtccgcatgatcaccaacccgactctcgatccgggcgtgatcttcggttatctaactcgcgccccattcggcggcgaaggctggatcatcggtgtgaactccatggaggacatcatcggtggccacatctggctgggtctgaccctgatcttcggtggcatctggcacgccatcaccaagcccttcggctgggtgcgtcgcgccttcatctggaacggtgaggcctacctgagctacagcctcggcgctctgagcttcatgagcttcatcgcctcggcctacatctggttcaacaacaccgcctatccctccgagttctggggccccaccaacgctgaggcatcccaggctcagagcttcaccttcctggtgcgtgaccagcgcctcggcgccaacatcggttccgccatgggccccaccggccttggtaagtacctgatgcgttcaccaaccggtgaaatcatcttcggtggtgaaaccatgcgtttctgggacttccgtggtccttggctggagcccctgcgtggccccaacggcctgagcctcgacaagctgcagaacgacattcagccctggcaagtgcgccgtgcggctgagtacatgacccacgctcccaacgcctcgatcaactccgtgggcggcatcatcaccgagcccaactcggtgaactacgtgaacctccgccagtggctgggtgcaacgcagttcgtgcttgccttcttcttcctggttggtcacctctggcacgccggccgcgcccgcgcagctgctgctggctttgagaaaggcatcgaccgcaaagctgagcctgtgctcggcatgcccgacctcgactga

SEQ ID NO: 42 - DNA, 61 bp, artificial sequence
  synthetic construct
ggtacagaccgcccaggccgagaacggcagagctgatcaggtgcagaacaccgaccacgaa

SEQ ID NO: 43 - DNA, 1011 bp, Synechococcus sp. WH8102
atggctccaccaactggtttttcgaagacgaaatcgcagctgcctgaggacttccctgtgagcgacgcacgtcagctgctgggcatgaaaggtgcctccggcacctccaacatctggaagctgcggctgcagctgatgaaaccggtcacctggatccccttgatctggggtgtgatctgcggtgctgccgctagtggcaactaccagtggaagctggaccacgtgctcgcggctttcgcatgcatgttgatgagcggccccctgctggcgggcttcacccaaaccatcaacgactattacgaccgcgatatcgacgcgatcaacgagccgtatcggccgattccatccggagccattccgttgggacaggtgaagcttcagatctggctgctgctgatcgctggcttggcggtgtcctacggcctcgacatctgggccaaccacagcactccggtggtgttcctgctggccctcggagggtctttcgtcagttacatctattcagctccaccgctgaagctgaagcagaacggttggcttgggaattacgcccttggtgccagctacatcgctctgccttggtgggcaggccaggccctgttcggccaactgacctggagcacggctctgctgacccttgcctacagcctcgccggactgggcattgccgtagtgaatgatttcaagagcgttgagggggaccgggaactggggcttcagtccttacccgttgtgttcgggatcaaaacggccagttggatcagcgctgggatgatcgacatcttccagttggccatggttgccgttctcattgccatcggtcagcatttcgctgctgttctgctggtgctgttgatcgtgccccagatcaccttccaggacatctggctgctgcgggacccagttgaatttgacgtcaaataccaagccagcgctcaaccctttctcgtgctcggcatgctggtgacggcgctggccgtgggccacagcccactcacccagctgatgtga

SEQ ID NO: 44 - DNA, 61 bp, artificial sequence
  synthetic construct
ccagtccggcgaggctgtaggcaagggtcagcagagccgtgctccaggtcagttggccgaa

SEQ ID NO: 45 - DNA, 789 bp, artificial sequence
  synthetic construct
atgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataa

SEQ ID NO: 46 - DNA, 408 bp, artificial sequence
  synthetic construct
ctgtctcttatacacatctagcgtctagacctagaggatccgggtaccgagctcgaattcgagctccccaatcctcgtgatgatcagtgatggaaaaagcactgtaattcccttggtttttggctgaaagtttcggactcagtagacctaagtacagagtgatgtcaacgccttcaagctagacgggaggcggcttttgccatggttcagcgatcgctcctcatcttcaataagcagggcatgagccagcgttaagcaaatcaaatcaaatctcgcttctgggcttcaataaatggttccgattgatgataggttgattcatgcaagcttggagcacaggatgacgcctaacaattcattcaagccgacaccgcttcgcggcgcggcttaattcaggagttaaacatc

SEQ ID NO: 47 - DNA, 266 bp, Synechococcus E14860
cggccgctactaaagcctgatttgtcttgatagctgctctgcctttgggcaggggcttttttctgtctgccattcttgaggatggcggactctttcccttttgctctacgcccatgaatgcgatcgcagtctcccctgtccagcacgttggagtgattggtggtggccagttagcttggagtctggcaccagcagcgcaacagttggggatgtcgctgcacgttcaaacacccaatgatcacgacccagcagtagcgatggagctc

SEQ ID NO: 48 - DNA, 50 bp, artificial sequence
  synthetic construct
gcctcccataacgggaggcttttttgcctcccataacgggaggctttttt

SEQ ID NO: 49 - DNA, 1825 bp, artificial sequence
  synthetic construct
gtcgacacatctatgacttggccttgatgacccaaaaagggtttgatgctgagggaatgaaagccttcattgagcgttctaatgcggtcttgacggcgttgacgactcgccagtgaagggtttaggggaactctaaacttcacagaacgtcatcctagctatctcgccctcagacgtgagcctcgctaagctgagggcgacgatccatttcgtggtgtggggtgaggaaatagtccatatcccgcagacaggccgcgaggcgtctggtggtgtaggcattcccaccagggaggagaagttccggctcatcacgcctcccataacgggaggcttttttgcctcccataacgggaggcttttttctgtctcttatacacatctagcgtctagacctagaggatccgggtaccgagctcgaattcgagctccccaatcctcgtgatgatcagtgatggaaaaagcactgtaattcccttggtttttggctgaaagtttcggactcagtagacctaagtacagagtgatgtcaacgccttcaagctagacgggaggcggcttttgccatggttcagcgatcgctcctcatcttcaataagcagggcatgagccagcgttaagcaaatcaaatcaaatctcgcttctgggcttcaataaatggttccgattgatgataggttgattcatgcaagcttggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataacggccgctactaaagcctgatttgtcttgatagctgctctgcctttgggcaggggcttttttctgtctgccattcttgaggatggcggactctttcccttttgctctacgcccatgaatgcgatcgcagtctcccctgtccagcacgttggagtgattggtggtggccagttagcttggagtctggcaccagcagcgcaacagttggggatgtcgctgcacgttcaaacacccaatgatcacgacccagcagtagcgatggagctc

SEQ ID NO: 50 - DNA, 1797 bp, artificial sequence
  synthetic construct
gtcgacacatctatgacttggccttgatgacccaaaaagggtttgatgctgagggaatgaaagccttcattgagcgttctaatgcggtcttgacggcgttgacgactcgccagtgaagggtttaggggaactctaaacttcacagaacgtcatcctagctatctcgccctcagacgtgagcctcgctaagctgagggcgacgatccatttcgtggtgtggggtggtacagaccgcccaggccgagaacggcagagctgatcaggtgcagaacaccgaccacgaagcctcccataacgggaggcttttttgcctcccataacgggaggcttttttctgtctcttatacacatctagcgtctagacctagaggatccgggtaccgagctcgaattcgagctccccaatcctcgtgatgatcagtgatggaaaaagcactgtaattcccttggtttttggctgaaagtttcggactcagtagacctaagtacagagtgatgtcaacgccttcaagctagacgggaggcggcttttgccatggttcagcgatcgctcctcatcttcaataagcagggcatgagccagcgttaagcaaatcaaatcaaatctcgcttctgggcttcaataaatggttccgattgatgataggttgattcatgcaagcttggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataacggccgctactaaagcctgatttgtcttgatagctgctctgcctttgggcaggggcttttttctgtctgccattcttgaggatggcggactctttcccttttgctctacgcccatgaatgcgatcgcagtctcccctgtccagcacgttggagtgattggtggtggccagttagcttggagtctggcaccagcagcgcaacagttggggatgtcgctgcacgttcaaacacccaatgatcacgacccagcagtagcgatggagctc

SEQ ID NO: 51 - DNA, 1797 bp, artificial sequence
  synthetic construct
gtcgacacatctatgacttggccttgatgacccaaaaagggtttgatgctgagggaatgaaagccttcattgagcgttctaatgcggtcttgacggcgttgacgactcgccagtgaagggtttaggggaactctaaacttcacagaacgtcatcctagctatctcgccctcagacgtgagcctcgctaagctgagggcgacgatccatttcgtggtgtggggtccagtccggcgaggctgtaggcaagggtcagcagagccgtgctccaggtcagttggccgaagcctcccataacgggaggcttttttgcctcccataacgggaggcttttttctgtctcttatacacatctagcgtctagacctagaggatccgggtaccgagctcgaattcgagctccccaatcctcgtgatgatcagtgatggaaaaagcactgtaattcccttggtttttggctgaaagtttcggactcagtagacctaagtacagagtgatgtcaacgccttcaagctagacgggaggcggcttttgccatggttcagcgatcgctcctcatcttcaataagcagggcatgagccagcgttaagcaaatcaaatcaaatctcgcttctgggcttcaataaatggttccgattgatgataggttgattcatgcaagcttggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataacggccgctactaaagcctgatttgtcttgatagctgctctgcctttgggcaggggcttttttctgtctgccattcttgaggatggcggactctttcccttttgctctacgcccatgaatgcgatcgcagtctcccctgtccagcacgttggagtgattggtggtggccagttagcttggagtctggcaccagcagcgcaacagttggggatgtcgctgcacgttcaaacacccaatgatcacgacccagcagtagcgatggagctc

SEQ ID NO: 52 - DNA, 2791 bp, artificial sequence
  synthetic construct
cattaatgaatattccgatagattagtgaaaccatcaaagcggatgaagagattcgaactctcgaccctctccttggcaaggagatgctctaccactgagctacatccgcaaatttgccgcagacctcgtccgtcggccaatgcatcatggactaccaacggtcccttggtcaagactcaggacggcctgcactgctaggggatgaggccaaatgcatggccacagctggaattcgcgccagtcacggaagcggaacatggattgactgaatcccgtgtaataagccgatatgacccttcgtcggaagccattatccacattccttccgtggcaactctcaccaccgtcgcaggcggtacttccaatcccgactacatcgaagtttccgagctcaaggattggttccttgacgccaaggaaggaaacgacgaggtcgttgtcgaagagacctcaactggttttgagctctatggcgcaggcgactcagacaccctcaccgcaatgggtgacgttgctgacgccatgatcaaaggcggtgcagctgctgactacatcaccatcaagggtgcaaccacaaacacttcggtttacggtggtaaggccgctgacagcatcactttcgaccgagctgttgttggtggagttgtctacggagacaccaataaggacactgaaatcactttcaccgacaaggtgagtggtggaaccatcgttgacggtggtgctgatgatgactccctcacgttcaacaagcggatcactagtgtgaccgttaggggcggtgcagcccgcgacgcgatcagtgttgctgaatcactggattctttagttgacgctggtggagataatgacaacctatctatctcaggttctcactccaacctgatcgcgaagggtggcgagggtgctgatacgctcgatcttactcttgcgggaactggcaacaggttctacggcggcaaggacaacgattcaatcaagatcgatacagccgctgcagtagctgttcacggtgataatgataacgacacaattgaaattgctgatagtgttgtttcaggtgcaagcgtattcggtggcgatggagctgacacactttccctgactgccgcccgagctggttcagaattagtagccaagggtaattccggcaacgacaaaatcgatggtgctacctctgagtccgatgaaaccattttcggtggacaaggtaatgacaccatcattagtagtgccgatggatctcgtacgtactacggtgacaaaggcgacgacgtcatcagcattggcacaaatgaagccagcatggtttcgggcggcgaaggtgctgatgactcaattaacgtcaacactgtagttacggctgctgatgaaaagttccacactgtcattggtggtgctggcgtagacacaattgtcgcagcaggttctacagatgctaaatacgcaactagccttcagtattcatccttcgctgaattcttcaccgctggtgatgtcgtcgactcaatcactgtcggtgatggcacttacgtaaaagcaaatgtcgctgaggcattgtctttcatcgatattgactcgttcgatcgagttacgatgagcgctggaacagatggtaagcgtactatcgcggccgaaggtctgatcatcgccactacagatgcggtgaccaccggttcatcaatcgtcttcgacagcagtgcagaggactacatcgctggtattgacctctccgcaagcgcaaccactgcaggttccttgatcgataactctgcaggtaacggtgccactgatcagggaatgatcctgaagggtactgaaggtgacaacaccattttgggtggtgatggcgctgatcaaatcactggtggatccggtggtgacagcctcaccggtggtgaaggagctgacacgattgatgctggtactgaaggtaccgacattcttgttggtggtgatggagatgactacctcgatctgaacaccgacctttctaaagacgacctcatcactggtggtgacggtactgataccatcgctttcagtcacaaatctgcctccaccaacattctcgacagagtgtctgaagtggaagtcgtcaaactggaaaatgcaaaagacaacgcatccatcacgctcctcgatacaacaattgcatctgacggcaagagcctgacagttacgaccaacaatgcaagcttcacaggcaagctcaccttcaacgcaagcgctgaaactgatggttcagtgaatgtcactggcggtgcctccgctgacaccattacaggttcagctggcgctgacacctttaatggtggcggtggtgttgacagcatcactggtggtcttggaattgatttctacgacttctcaacagttgcaaactggggagataccattaccgattacggaaagagcactgctacggctaatgctcaaaacaccacagctctctcgaacgaggccatttctcttaacggtgaagctctggccttcagtgatgctgcaatttcatcaaatgcaaattcagccattgttggttcctacactccaccatctggagacaacgcatccaccttcaacgcaactgccttgaagtctggtactacagccgcccctgcagtcgtggatcaggcttatgcacagttcctgtacaacacagacaccggtgtcctcagcttcgacgctgacggaactggcactaacaacacggcagttactgttgcaactctattaaacggagctactgcgcctacattgacttcaactgatcttgtgattttcgcttga

SEQ ID NO: 53 - DNA, 260 bp, Chlamydomonas reinhardtii Y16833
gcagttgggtcaggggctggcgacgcgctgctgacgcgcaagtgaatggcccaacaagtcgcctcgcggtcgctgtcggcgccaaacccgcagctgcatccaccagattcacttgttagatcgacctaggttgcgggaccggaggcggctcgctgtgcaagcgcggtgacctcgtacggcggcatggatcgccatctcgattcgcgcggcagaatcgggccccgcgcacatttaagccgcgggcgagactcatttcgtta

SEQ ID NO: 54 - DNA, 1181 bp, artificial sequence
  synthetic construct
gccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggatggccaagctgaccagcgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactaaccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcttaattaactcgagggggggcccggtacc

SEQ ID NO: 55 - DNA, 1550 bp, artificial sequence
  synthetic construct
gccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggatggccaagctgaccagcgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactaaccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcttaattaactcgagggggggcccggtaccatactcatcgatagctatcgtagaaaggaggtcaagaacggccgcctggtaaatcgatccaggcggccgttcttgacctccttatactcatcgatagctatcgtagagcgtcccggcacctgcgctgctagctgatgtcaccccttcctggggcgtgatgactggcagcgcaccaaaaaaactcggtgtttatcaacaccaccttattctcgtggtctgggcgggagggttgagagtctgcaaagctcagcgattgacgtgccctttgcgggcagcagtgcctgaccgtgaagcacggcaaggtggcatacgaggtgtgagcacaaggaggaaagcactctgggcagtgcatgcgatcgtatgcgatgt

SEQ ID NO: 56 - DNA, 22 bp, artificial sequence
  synthetic construct
acatcgcatacgatcgcatgca

SEQ ID NO: 57 - DNA, 262 bp, Chlamydomonas reinhardtii
gcgtcccggcacctgcgctgctagctgatgtcaccccttcctggggcgtgatgactggcagcgcaccaaaaaaactcggtgtttatcaacaccaccttattctcgtggtctgggcgggagggttgagagtctgcaaagctcagcgattgacgtgccctttgcgggcagcagtgcctgaccgtgaagcacggcaaggtggcatacgaggtgtgagcacaaggaggaaagcactctgggcagtgcatgcgatcgtatgcgatgt

SEQ ID NO: 58 - DNA, 345 bp, artificial sequence
  synthetic construct
atactcatcgatagctatcgtagaaaggaggtcaagaacggccgcctggtaaatcgatccaggcggccgttcttgacctccttgcgtcccggcacctgcgctgctagctgatgtcaccccttcctggggcgtgatgactggcagcgcaccaaaaaaactcggtgtttatcaacaccaccttattctcgtggtctgggcgggagggttgagagtctgcaaagctcagcgattgacgtgccctttgcgggcagcagtgcctgaccgtgaagcacggcaaggtggcatacgaggtgtgagcacaaggaggaaagcactctgggcagtgcatgcgatcgtatgcgatgt

SEQ ID NO: 59 - DNA, 260 bp, Chlamydomonas reinhardtii
ccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttg

SEQ ID NO: 60 - DNA, 32 bp, artificial sequence
  synthetic construct
ggccccaaccgcgccaagtggctgggccctac

SEQ ID NO: 61 - DNA, 25 bp, artificial sequence
  synthetic construct
gactacggctgggacaccgccggtc

SEQ ID NO: 62 - DNA, 1382 bp, Chlamydomonas reinhardti
gtaagttcccgcctcgcctcgccctcatccaccccgattgcccccggacagccctggcgcgcttggtggcattgctgacaggccgccgttccgcaggactcctcacacaacacacggcacatggcacatagccgcgctttccttgctgactaccgtaggcgggtgtatgcgcgctgcaagccagtgcatgtaggcatgcagatccttgggtgctttccacgttcccccaccggatgttgcctcccgcatccctctccttacttccatcccataccagcgtgtgcaaggatacaatcaaggcccaaagcttctcgtgtggctgctgatgtacggaactgtgccagtgcgagggacccgaccggatccaagttcggactgaacacacatcgacctggctccgttccaggtgggtgcattttgtgggtttcgcgttgtcacggcgaatggcagcagcctagttgcgagctggaacacgtccgcccctgcaagcggcagcagtctccacgacactcggactccgtttggcgtgccgaatgcggagcagcgtggagcgcgcaccggtgtctcgtctcgaaggtaacaagtaaccgcattgccgctgacactttattgctagcctggagctcgcgacggcgtcttgcattcgcattcactagcaagcacttcgctgatcgacctcccctgatctgtcaggctagtcggggaaacccgcgtgccctctttcgctggtgggcctgcccctgaatttgcactatgtcttgagcttgggttatgacgcttgccccggggctcctgaggcaatgaggcagcaaccctgatcctgggcaaggtcctggggcgacggcgggccaaagttcaggacccgcggaccgcggtcgcggcagtagccgagtggaccccggacagcgccctctcgatgccaacaagactggtaggacgtgcgatacaacttcttttgccccaagccaacctggcctttgagcgtgacgaacctctggcaagactgcttcttgctgacagcagaaacgccagtctgcttccagagatacaaaattgtatacttaatgtgcatgatagcatgtttcagcttcgctttggggcattcaagttgccttcggtactttgcatccggcttcgcttgtagccgttatggctcatttccacttcccgctttatgcattcagatagatttaacgcctgatatcaagactaattcgcgccgtctgcattggtgcatgcagcgcgccaaacgcagaggaccgctcgctgcaaggccgtacacctcatcgcggatgaggagcgcgtttccagtcaaaagcttctgcttccgctttgccaaagttgtctgacccttcagtccgaacaacccacttacaatcatg